(19) United States
(12) Patent Application Publication
(10) Pub. No.: US 2018/0042845 A1
(43) Pub. Date: Feb. 15, 2018

(54) PREPARATIONS OF CANNABIS EMULSIONS AND METHODS THEREOF

(71) Applicant: ONE WORLD CANNABIS LTD., Petach Tikva (IL)

(72) Inventors: Alon SINAI, PETACH TIKVA (IL); Ziv TURNER, PETACH TIKVA (IL); Yehuda BARUCH, GEDERA (IL)

(73) Assignee: ONE WORLD CANNABIS LTD., PETACH TIKVA (IL)

(21) Appl. No.: 15/708,187

(22) Filed: Sep. 19, 2017

Related U.S. Application Data
(63) Continuation-in-part of application No. PCT/IL2016/050289, filed on Mar. 17, 2016.
(60) Provisional application No. 62/135,185, filed on Mar. 19, 2015.

(51) Int. Cl. A61K 9/107; A61K 31/352; A61K 47/10; A61K 47/24; A61K 47/18; A61K 47/14; A61K 9/00; A61K 38/18

(52) U.S. Cl. CPC A61K 9/1075; A61K 9/006; A61K 9/0002; A61K 31/352; A61K 38/18; A61K 47/24; A61K 47/183; A61K 47/14; A61K 47/10

(57) ABSTRACT

The present invention discloses a cannabis based emulsion formulation for use in various medical conditions and optionally with various pharmaceutical or nutraceutical compositions, wherein the oily fraction used contains about 50% cannabinoids. The present invention further discloses methods of manufacturing and uses of the aforementioned composition.

### CROSS REFERENCE TO RELATED APPLICATIONS

This application is a Continuation-in-Part of PCT International Application No. PCT/IL2016/050289, filed Mar. 17, 2016, which claims priority from U.S. Provisional Application No. 62/135,185, filed Mar. 19, 2015. Both of these applications are hereby incorporated by reference in their entirety.

### FIELD OF THE INVENTION

The present disclosure relates to novel compositions and methods for administration of pharmaceutical formulations. More particularly the current invention pertains to an emulsion comprising enriched concentrations of Tetrahydrocannabinol (THC), cannabidiol (CBD) or derivatives thereof, useful as a novel administration route for the treatment of various medical conditions.

### BACKGROUND OF THE INVENTION

Cannabis plants produce a group of chemicals called cannabinoids, which produce mental and physical effects when consumed. Cannabinoids are a group of 21-carbon-containing terpenophenolic compounds produced by Cannabis species. Two of the most prominent cannabinoids are Cannabidiol (CBD) and Tetrahydrocannabinol (THC).

Administered orally, THC is almost completely absorbed (90-95%) after a single oral dose. However, due to the combined effect of first pass hepatic metabolism and high lipid solubility, only about 10-20% of an administered dose reaches systemic circulation with highly variable maximal concentrations.

Several patent documents recite emulsions containing cannabinoids; U.S. Pat. No. 6,383,513 discloses a nasal administration of 150 μl (per nostril) containing a dose of 1 mg of THC, and having a particle average size of 250 nm. The drug is typically dissolved in the oil phase at a concentration of 0.1 to 20% w/v.

US Patent application 2007/0104741 discloses 10 mg of THC in a formulation of 260 mg fill weight, i.e. at a concentration of 3.8% v/v, for oral administration.

Being a highly lipophilic, essentially water insoluble, CBD and THC are difficult to formulate in relatively high concentration in pharmaceutical formulations, without having to increase the oily fraction. However, some administration routes are limited in the amount of their single serving, and therefore are limited in the total amount of cannabinoids which may be administered in a single dose.

In view of the above, it is still a long felt and unmet need for a cannabis emulsion having an enriched cannabinoid concentration.

### SUMMARY OF THE INVENTION

It is thus an object of the present invention to provide a composition comprising phospholipids, or derivatives thereof, and an oily fraction, the composition is formulated as an emulsion, wherein the oily fraction contains about 50% cannabinoids.

It is a further object of the present invention to provide the composition as defined in any of the above, wherein said composition comprises small-size particles in a diameter range of about 0.1 μm to 1 μm configured to be absorbed through mucosal membranes.

It is a further object of the present invention to provide the composition as defined in any of the above, wherein said particles diameter is in the range of about 0.4 μm to 0.45 μm.

It is a further object of the present invention to provide the composition as defined in any of the above, wherein said composition has instability index in the range of 0.2 to 0.5.

It is a further object of the present invention to provide the composition as defined in any of the above, wherein said composition has a pH range of about 5.5 to about 7.5.

It is a further object of the present invention to provide the composition as defined in any of the above, wherein said composition is administered in a manner selected from the group consisting of: intranasal, sublingual, buccal, transdermal, oromucosal, suppository, rectal, intramuscular, inhalational aerosol, transdermal, intravenous, oral, topical and any combination thereof.

It is a further object of the present invention to provide the composition as defined in any of the above, wherein said composition comprises a variety of molecules specifically fatty molecules, without sediments or any solid particles.

It is a further object of the present invention to provide the composition as defined in any of the above, wherein said composition is formulated in a dosage form selected from the group consisting of liquid, solid, gas, oral, pill, tablet, capsule, caplet, buccal, sub-lingual, orally-disintegrating, thin film, liquid solution, suspension, powder or liquid or solid crystals, pastes, inhalational, aerosol, inhaler, nebulizer, smoking, vaporizer, spray, syrup, parenteral, intradermal, intramuscular, intraosseous, intraperitoneal, intravenous, subcutaneous, topical, cream, gel, liniment or balm, lotion, ointment, drops, skin patch, vaginal, suppository, pessary, rectal and any combination thereof.

It is a further object of the present invention to provide the composition as defined in any of the above, wherein said composition comprises a mixture of at least two cannabinoids.

It is a further object of the present invention to provide the composition as defined in any of the above, wherein said composition comprises predetermined ratio of said cannabinoids.

It is a further object of the present invention to provide the composition as defined in any of the above, wherein said cannabinoid is extracted from cannabis; said cannabis is selected from the group consisting of: Cannabis sativa, Cannabis indica, Cannabis ruderalis, and any combination thereof.

It is a further object of the present invention to provide the composition as defined in any of the above, wherein said cannabinoid is selected from the group consisting of cannabinoid-type, cannabinoid derivative, cannabis extract or fraction thereof and any combination thereof.

It is a further object of the present invention to provide the composition as defined in any of the above, wherein said cannabinoid is selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

It is a further object of the present invention to provide the composition as defined in any of the above, wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

It is a further object of the present invention to provide the composition as defined in any of the above, wherein the cannabidiol (CBD) or a derivative thereof is selected from the group consisting of CBD, CBDV, CBDA and any combination thereof.

It is a further object of the present invention to provide the composition as defined in any of the above, wherein said THC or a derivative thereof is selected from the group consisting of natural THC or a derivative thereof produced in the body of humans and animals, THC or a derivative thereof extracted from plants, synthetic THC or a derivative thereof, and any combination thereof.

It is a further object of the present invention to provide the composition as defined in any of the above, wherein said CBD or a derivative thereof is selected from the group consisting of natural CBD or a derivative thereof produced in the body of humans and animals, CBD or a derivative thereof extracted from plants, synthetic CBD or a derivative thereof, and any combination thereof.

It is a further object of the present invention to provide the composition as defined in any of the above, wherein said composition comprising Tetrahydrocannabinol (THC) and Cannabidiol (CBD), or an extract comprising said Tetrahydrocannabinol (THC) and Cannabidiol (CBD), said ratio of said THC:CBD is about 40:10% w/w.

It is a further object of the present invention to provide the composition as defined in any of the above, wherein said composition is configured to be stable for at least 3 months at up to 40° C.

It is a further object of the present invention to provide the composition as defined in any of the above, wherein said composition comprises about 20% cannabis oil.

It is a further object of the present invention to provide the composition as defined in any of the above, wherein said composition comprises an oily phase and a water phase in a ratio of about 20% to about 80%, respectively.

It is a further object of the present invention to provide the composition as defined in any of the above, wherein said oily phase comprises about 0.3% of phospholipids.

It is a further object of the present invention to provide the composition as defined in any of the above wherein said composition comprises cannabis, surfactant, glycerol, antioxidants and water.

It is a further object of the present invention to provide the composition as defined in any of the above wherein said cannabis is cannabis oil, said antioxidants are tocopherol or EDTA or any combination thereof; said surfactant is phospholipids or tween 80 or any combination thereof.

It is a further object of the present invention to provide the composition as defined in any of the above, wherein said oily phase comprises cannabis oil, egg phospholipids, soya phospholipids, sodium oleate and tocopherol and wherein said water phase comprises glycerol, EDTA, and water.

It is a further object of the present invention to provide the composition as defined in any of the above, wherein at least one of the following holds true:
a. said emulsion has an instability index in the range of about 0.236 to about 0.386; and
b. said emulsion prepared in sterile environment has an instability index of about 0.442.

It is a further object of the present invention to provide the composition as defined in any of the above, wherein at least one of the following holds true:
a. said emulsion is with a particle diameter in the range of about 0.427 μm to about 0.493 μm; and
b. said emulsion prepared in sterile environment is with a particle diameter of about 0.414 μm.

It is a further object of the present invention to provide the composition as defined in any of the above, wherein at least one of the following holds true:
a. said cannabinoids are selected from the group consisting of cannabidiol (CBD) or a derivative thereof, Tetrahydrocannabinol (THC) or a derivative thereof, and any combination thereof;
b. final concentration of said THC or a derivative thereof, in said emulsion is in the range of about 80 to about 100 mg/ml;
c. a single dose of about 0.15 ml of said cannabis emulsion contains a dose of about 12 to about 15 mg THC;
d. the ratio of said oily fraction and said phospholipid is between 8:1 and 17:1;
e. said oily fraction is in the range of about 5% to about 50%; and
f. said oily fraction is in the range of about 10% to about 30%.

It is a further object of the present invention to provide the composition as defined in any of the above, wherein at least one of the following holds true:
a. said oily fraction is selected from the group consisting of cannabis oil, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and any combination thereof;
b. said composition further comprising antioxidants in the range of about 0.01% to about 0.1% w/v, and selected from the group consisting of ethanol, polyethylene glycol 300, polyethylene glycol 400, propylene glycol, propylene carbonate, N-methyl-2-pyrrolidones, dimethylacetamide, dimethyl sulfoxide, hydroxypropyl-β-cyclodextrins, sulfobutylether-β-cyclodextrin, α-cyclodextrin, HSPC phospholipid, DSPG phospholipid, DMPC phospholipid, DMPG phospholipid, ascorbyl palmitate, butylated hydroxy anisole, butylatedhydroxy anisole, propyl gallate, α-tocopherol, γ-tocopherol and any combination thereof;
c. said composition further comprising co-surfactants in the range of about 1% to about 10% w/v, and selected from the group consisting of glycerol, sodium stearate, potassium laurate, sodium dodecyl sulfate, sodium sulfosuccinate, polyglycol, fatty acid esters, quaternary ammonium salts, amine hydrochlorides and any combination thereof;
d. said composition further comprising chelating agents in the range of about 0.01% to about 0.5% w/v, and said chelating agents are selected from the group consisting of Ethylenediaminetetraacetic acid (EDTA), phosphoric acid, polyphosphates, polysaccharides, citric acid and any combination thereof;
e. said phospholipids are selected from the group consisting of phosphatidylcholine, diphosphatidylglycerol, phosphatidylethanolamine, phosphatidylserine, phosphatidylinositol, sphingomyelin, PEG phospholipid and any combination thereof; and
f. said phospholipids, or derivatives thereof, are derived of naturally-occurring food sources selected from the group consisting of poultry eggs, soya, rapeseed, sunflower, cattle milk, fish eggs and any combination thereof; or said phospholipids, or derivative thereof, are produced by a synthetic route.

It is a further object of the present invention to provide the composition as defined in any of the above, wherein at least one of the following holds true:
a. particle size of said emulsion is in the range of about 75 nm to about 150 nm;
b. particle size of said emulsion is in the range of about 100 nm to about 400 nm
c. said composition's pH is in the range of about 6.5 to about 7.5; and
d. said composition's pH is in the range of about 7.0 to about 7.5.

It is a further object of the present invention to provide the composition as defined in any of the above, wherein at least one of the following holds true:
a. said composition's osmolarity is in the range of about 200 milliosmolar/liter to about 500 milliosmolar/liter;
b. said composition's osmolarity is in the range of about 270 milliosmolar/liter to about 380 milliosmolar/liter;
c. said composition is stable at room temperature for about 3 months to about 12 months;
d. said composition is stable at fridge temperature for about 6 months to about 24 months;
e. said composition is stable at about 40 degrees Celsius temperature for about 2 months to about 6 months; and
f. stability of said composition is measured using a technique selected from the group consisting of measuring drop size, light scattering, focused beam reflectance measurement, centrifugation, rheology and any combination thereof.

It is a further object of the present invention to provide the composition as defined in any of the above, wherein at least one of the following holds true:
a. said composition is for oral administration in a formulation selected from a group of preparations consisting of syrup, drops, solution, suspension, tablet, bolus, troche, capsule and any combination thereof;
b. said composition is for topical administration in a formulation selected from a group of preparations consisting of cream, ointment lotion, foam, transdermal patch and any combination thereof;
c. wherein said composition is to be administered in combination with at least one pharmaceutical agent; and
d. said composition is to be administered in combination with at least one nutraceutical agent.

T It is a further object of the present invention to provide the composition as defined in any of the above, wherein at least one of the following holds true:
a. said CBD or said derivative thereof interacts with at least one receptor; said receptor is selected from a group consisting of Cannabinoid receptor type 1 (CB1), Cannabinoid receptor type 2 (CB2), any other cannabinoid receptor, and any combination thereof;
b. said THC or said derivative thereof interacts with at least one receptor; said receptor is selected from a group consisting of Cannabinoid receptor type 1 (CB1), Cannabinoid receptor type 2 (CB2), any other cannabinoid receptor, and any combination thereof;
c. said composition additionally comprises inactive ingredients selected from a group consisting of antiadherents, binders, coatings, disintegrants, flavours, colours, lubricants, glidants, sorbents, preservatives, sweeteners, and any combination thereof;
d. said composition is in a sustained release dosage form; said sustained release dosage form is selected from a group consisting of drug polymer conjugates, microencapsulation, controlled-release tablet coating, and any combination thereof;
e. said composition is nonpsychoactive; and
f. said composition is administered once, twice, three or four times through the day.

It is a further object of the present invention to provide the composition as defined in any of the above, wherein at least one of the following holds true:
a. said oily fraction is cannabis oil obtained from at least one cannabis plant;
b. said CBD or derivative thereof is produced by a synthetic route; and
c. said THC or derivative thereof is produced by a synthetic route.

It is a further object of the present invention to provide the composition as defined in any of the above, wherein said cannabis plant is a CBD rich strain, or wherein said cannabis plant is a THC rich strain.

It is a further object of the present invention to provide the composition as defined in any of the above, wherein said CBD rich strain is selected from a group consisting of Golan, Avidekel, Fedora 17, ACDC, and any combination thereof; or wherein said cannabis plant is a THC rich strain; said THC rich strain is selected from a group consisting of Everest, Black Destroyer, Critical Neville Haze, Mataro Blue, LSD OG Kush, Pineapple Chunk, Blue Monster Holk, Y Griega, Satori, Tutankhamon, and any combination thereof.

It is a further object of the present invention to provide the composition as defined in any of the above, wherein at least one of the following holds true:
a. said composition is formulated for administration of about 5 mg to about 15 mg THC per dosage unit;
b. said composition further comprises an additional lipophilic solvent or suspension carrier;
c. said lipophilic solvent or suspension carrier are selected from a group consisting of medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, vegetable oil, and any combination thereof;
d. further comprising pH adjusting agents, selected from the group consisting of disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and any combination thereof; and
e. further comprising osmotic agents, selected from the group consisting of glycerin, glucose and sucrose, sorbitol, sodium phosphate and any combination thereof.

It is a further object of the present invention to provide the composition as defined in any of the above, wherein at least one of the following holds true:
a. said composition further comprising flavoring agents, selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof; and
b. said composition further comprising preservatives, selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

It is a further object of the present invention to provide the composition as defined in any of the above, wherein at least one of the following holds true:
a. said composition comprises a mixture of at least two cannabinoids;
b. said composition comprises predetermined ratio of said cannabinoids;
c. said cannabinoid is extracted from cannabis; said cannabis is selected from the group consisting of: Cannabis sativa, Cannabis indica, Cannabis ruderalis, and any combination thereof;
d. said cannabinoid is selected from the group consisting of cannabinoid-type, cannabinoid derivative, cannabis extract or fraction thereof and any combination thereof; and
e. said cannabinoid is selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

It is a further object of the present invention to provide the composition as defined in any of the above, wherein at least one of the following holds true:
a. the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof;
b. the cannabidiol (CBD) or a derivative thereof is selected from the group consisting of CBD, CBDV, CBDA and any combination thereof;
c. said THC or a derivative thereof is selected from the group consisting of natural THC or a derivative thereof produced in the body of humans and animals, THC or a derivative thereof extracted from plants, synthetic THC or a derivative thereof, and any combination thereof; and
d. said CBD or a derivative thereof is selected from the group consisting of natural CBD or a derivative thereof produced in the body of humans and animals, CBD or a derivative thereof extracted from plants, synthetic CBD or a derivative thereof, and any combination thereof.

It is a further object of the present invention to provide the composition as defined in any of the above, wherein at least one of the following holds true:
a. said composition comprising Tetrahydrocannabinol (THC) and Cannabidiol (CBD), or an extract comprising said Tetrahydrocannabinol (THC) and Cannabidiol (CBD), said ratio of said THC:CBD is about 40:10% w/w;
b. said composition is configured to be stable for at least 3 months at up to 40° C.;
c. said composition comprises about 20% cannabis oil;
d. said composition comprises an oily phase and a water phase in a ratio of about 20% to about 80%, respectively;
e. said oily phase comprises about 0.3% of phospholipids; said composition comprises cannabis, surfactant, glycerol, antioxidants and water; and
f. said oily phase comprises cannabis oil, egg phospholipids, soya phospholipids, sodium oleate and tocopherol and wherein said water phase comprises glycerol, EDTA, and water.

It is a further object of the present invention to provide the composition as defined in any of the above wherein said cannabis is cannabis oil, said antioxidants are tocopherol or EDTA or any combination thereof; said surfactant is phospholipids or tween 80 or any combination thereof.

It is a further object of the present invention to provide the composition as defined in any of the above, wherein at least one of the following holds true:
a. said cannabinoids are selected from the group consisting of cannabidiol (CBD) or a derivative thereof, Tetrahydrocannabinol (THC) or a derivative thereof, and any combination thereof;
b. final concentration of said THC or a derivative thereof, in said emulsion is in the range of about 80 to about 100 mg/ml;
c. a single dose of about 0.15 ml of said cannabis emulsion contains a dose of about 12 to about 15 mg THC;
d. the ratio of said oily fraction and said phospholipid is between 8:1 and 17:1;
e. said oily fraction is in the range of about 5% to about 50%; and
f. said oily fraction is in the range of about 10% to about 30%.

It is a further object of the present invention to provide the composition as defined in any of the above, wherein at least one of the following holds true:
a. said oily fraction is selected from the group consisting of cannabis oil, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and any combination thereof;
b. said composition further comprising antioxidants in the range of about 0.01% to about 0.1% w/v, and selected from the group consisting of ethanol, polyethylene glycol 300, polyethylene glycol 400, propylene glycol, propylene carbonate, N-methyl-2-pyrrolidones, dimethylacetamide, dimethyl sulfoxide, hydroxypropyl-β-cyclodextrins, sulfobutylether-β-cyclodextrin, α-cyclodextrin, HSPC phospholipid, DSPG phospholipid, DMPC phospholipid, DMPG phospholipid, ascorbyl palmitate, butylated hydroxy anisole, butylatedhydroxy anisole, propyl gallate, α-tocopherol, γ-tocopherol and any combination thereof;
c. said composition further comprising co-surfactants in the range of about 1% to about 10% w/v, and selected from the group consisting of glycerol, sodium stearate, potassium laurate, sodium dodecyl sulfate, sodium sulfosuccinate, polyglycol, fatty acid esters, quaternary ammonium salts, amine hydrochlorides and any combination thereof;
d. said composition further comprising chelating agents in the range of about 0.01% to about 0.5% w/v, and said chelating agents are selected from the group consisting of Ethylenediaminetetraacetic acid (EDTA), phosphoric acid, polyphosphates, polysaccharides, citric acid and any combination thereof;
e. said phospholipids are selected from the group consisting of phosphatidylcholine, diphosphatidylglycerol, phosphatidylethanolamine, phosphatidylserine, phosphatidylinositol, sphingomyelin, PEG phospholipid and any combination thereof;
f. said phospholipids, or derivatives thereof, are derived of naturally-occurring food sources selected from the group consisting of poultry eggs, soya, rapeseed, sunflower, cattle milk, fish eggs and any combination thereof; or said phospholipids, or derivative thereof, are produced by a synthetic route;
g. said composition's osmolarity is in the range of about 200 milliosmolar/liter to about 500 milliosmolar/liter;
h. said composition's osmolarity is in the range of about 270 milliosmolar/liter to about 380 milliosmolar/liter;
i. said composition is stable at room temperature for about 3 months to about 12 months;
j. said composition is stable at fridge temperature for about 6 months to about 24 months;
k. said composition is stable at about 40 degrees Celsius temperature for about 2 months to about 6 months;
l. stability of said composition is measured using a technique selected from the group consisting of measuring drop size, light scattering, focused beam reflectance measurement, centrifugation, rheology and any combination thereof.
m. wherein said composition is to be administered in combination with at least one pharmaceutical agent;
n. said composition is to be administered in combination with at least one nutraceutical agent.
o. said composition additionally comprises inactive ingredients selected from a group consisting of antiadherents, binders, coatings, disintegrants, flavours, colours, lubricants, glidants, sorbents, preservatives, sweeteners, and any combination thereof;
p. said composition is in a sustained release dosage form; said sustained release dosage form is selected from a group consisting of drug polymer conjugates, microencapsulation, controlled-release tablet coating, and any combination thereof;
q. said composition is nonpsychoactive;
r. said composition is administered once, twice, three or four times through the day;
s. said oily fraction is cannabis oil obtained from at least one cannabis plant;
t. said CBD or derivative thereof is produced by a synthetic route; and
u. said THC or derivative thereof is produced by a synthetic route.

It is a further object of the present invention to provide the composition as defined in any of the above, wherein said cannabis plant is a CBD rich strain, or wherein said cannabis plant is a THC rich strain.

It is a further object of the present invention to provide the composition as defined in any of the above, wherein said CBD rich strain is selected from a group consisting of Golan, Avidekel, Fedora 17, ACDC, and any combination thereof; or wherein said cannabis plant is a THC rich strain; said THC rich strain is selected from a group consisting of Everest, Black Destroyer, Critical Neville Haze, Mataro Blue, LSD OG Kush, Pineapple Chunk, Blue Monster Holk, Y Griega, Satori, Tutankhamon, and any combination thereof.

It is a further object of the present invention to provide the composition as defined in any of the above, wherein at least one of the following holds true:
a. said composition further comprises an additional lipophilic solvent or suspension carrier;
b. said lipophilic solvent or suspension carrier are selected from a group consisting of medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, vegetable oil, and any combination thereof;
c. further comprising pH adjusting agents, selected from the group consisting of disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and any combination thereof;
d. further comprising osmotic agents, selected from the group consisting of glycerin, glucose and sucrose, sorbitol, sodium phosphate and any combination thereof;
e. said composition further comprising flavoring agents, selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof; and
f. said composition further comprising preservatives, selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

It is a further object of the present invention to provide the composition as defined in any of the above, wherein said composition is prepared by the steps of:
a. combining phospholipids, or derivatives thereof, and an oily fraction, thereby obtaining an oily phase;
b. combining said oily phase with a water phase in an emulsifier, thereby obtaining pre-emulsion; and
c. transferring said pre-emulsion into a microfluidizer, thereby obtaining a cannabis composition in the formulation of an emulsion;
   further wherein said oily fraction contains about 50% cannabinoids.

It is a further object of the present invention to provide the composition as defined in any of the above, further prepared by steps of:
a. preparing an oil phase by mixing cannabis oil, soya phospholipids, sodium oleate and tocopherol and heating to 65° C. for 20 min;
b. preparing a water phase by mixing glycerol, EDTA and water and heating to 75° C. for 20 min;
c. adding the water phase to the oil phase and pre-mixing for 2 min at 27,000 RPM;
d. preparation of an emulsion from said mixture of step c with high-pressure homogenizer at air pressure of 150 Psi (process pressure of 45 kPsi), at room temperature;
e. measuring pH of said emulsion and optionally adjusting to pH in the range of about 5.5 to 7.5;
f. optionally, sterilizing said emulsion.

It is a further object of the present invention to provide the composition as defined in any of the above, for use in treating or preventing a medical condition in a subject.

It is a further object of the present invention to provide the use as defined in any of the above, wherein said composition comprises small-size particles in a diameter range of about 0.1 μm to 1 μm, configured to be absorbed through mucosal membranes.

It is a further object of the present invention to provide the use as defined in any of the above, wherein said composition has instability index in the range of 0.2 to 0.5.

It is a further object of the present invention to provide the use as defined in any of the above, wherein said composition has a pH range of about 5.5 to about 7.5.

It is a further object of the present invention to provide the use as defined in any of the above, wherein said composition comprises about 20% cannabis oil, said cannabis oil comprises THC:CBD ratio of about 40:10% w/w.

It is a further object of the present invention to provide the use as defined in any of the above, wherein said composition is administered in a manner selected from the group consisting of: intranasal, sublingual, buccal, transdermal, oromucosal, suppository, rectal, intramuscular, inhalational aerosol, transdermal, intravenous, oral, topical and any combination thereof.

It is a further object of the present invention to provide a method of treating or preventing a medical condition in a subject; said method comprising administrating to the subject a therapeutically effective amount of a composition comprising phospholipids, or derivatives thereof, and an oily fraction, said composition is formulated as an emulsion, wherein said oily fraction contains about 50% cannabinoids.

It is also an object of the present invention to provide the composition as mentioned above, wherein the cannabinoids are selected from the group consisting of cannabidiol (CBD) or a derivative thereof, Tetrahydrocannabinol (THC) or a derivative thereof, and any combination thereof.

It is also an object of the present invention to provide the composition as mentioned above, wherein final concentration of the THC or a derivative thereof, in the emulsion is about 100 mg/ml.

It is also an object of the present invention to provide the composition as mentioned above, wherein a single dose of about 0.15 ml of the cannabis emulsion contains a dose of about 15 mg THC.

It is also an object of the present invention to provide the composition as mentioned above, wherein the ratio of the oily fraction and the phospholipid is between 8:1 and 17:1.

It is also an object of the present invention to provide the composition as mentioned above, wherein the oily fraction is in the range of about 5% to about 50%.

It is also an object of the present invention to provide the composition as mentioned above, wherein the oily fraction is in the range of about 10% to about 30%.

It is also an object of the present invention to provide the composition as mentioned above, wherein the oily fraction is selected from the group consisting of cannabis oil, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and any combination thereof.

It is also an object of the present invention to provide the composition as mentioned above, further comprising antioxidants in the range of about 0.01% to about 0.1% w/v, and selected from the group consisting of ethanol, polyethylene glycol 300, polyethylene glycol 400, propylene glycol, propylene carbonate, N-methyl-2-pyrrolidones, dimethylacetamide, dimethyl sulfoxide, hydroxypropyl-β-cyclodextrins, sulfobutylether-β-cyclodextrin, α-cyclodextrin, HSPC phospholipid, DSPG phospholipid, DMPC phospholipid, DMPG phospholipid, ascorbyl palmitate, butylated hydroxy anisole, butylatedhydroxy anisole, propyl gallate, α-tocopherol, γ-tocopherol and any combination thereof.

It is also an object of the present invention to provide the composition as mentioned above, further comprising co-surfactants in the range of about 1% to about 10% w/v, and selected from the group consisting of glycerol, sodium stearate, potassium laurate, sodium dodecyl sulfate, sodium sulfosuccinate, polyglycol, fatty acid esters, quaternary ammonium salts, amine hydrochlorides and any combination thereof.

It is also an object of the present invention to provide the composition as mentioned above, further comprising chelating agents in the range of about 0.01% to about 0.5% w/v, and the chelating agents are selected from the group consisting of Ethylenediaminetetraacetic acid (EDTA), phosphoric acid, polyphosphates, polysaccharides, citric acid and any combination thereof.

It is also an object of the present invention to provide the composition as mentioned above, wherein the phospholipids are selected from the group consisting of phosphatidylcholine, diphosphatidylglycerol, phosphatidylethanolamine, phosphatidylserine, phosphatidylinositol, sphingomyelin, PEG phospholipid and any combination thereof.

It is also an object of the present invention to provide the composition as mentioned above, wherein the phospholipids, or derivatives thereof, are derived of naturally-occurring food sources selected from the group consisting of poultry eggs, soya, rapeseed, sunflower, cattle milk, fish eggs and any combination thereof.

It is also an object of the present invention to provide the composition as mentioned above, wherein the phospholipids, or derivative thereof, are produced by a synthetic route.

It is also an object of the present invention to provide the composition as mentioned above, wherein the average particle size of the emulsion is in the range of about 50 nm to about 400 nm.

It is also an object of the present invention to provide the composition as mentioned above, wherein particle size of the emulsion is in the range of about 75 nm to about 150 nm.

It is also an object of the present invention to provide the composition as mentioned above, wherein particle size of said emulsion is in the range of about 100 nm to about 400 nm.

It is also an object of the present invention to provide the composition as mentioned above, wherein the composition's pH is in the range of about 6.5 to about 7.5.

It is also an object of the present invention to provide the composition as mentioned above, wherein the composition's pH is in the range of about 7.0 to about 7.5.

It is also an object of the present invention to provide the composition as mentioned above, wherein the composition's osmolarity is in the range of about 200 milliosmolar/liter to about 500 milliosmolar/liter.

It is also an object of the present invention to provide the composition as mentioned above, wherein the composition's osmolarity is in the range of about 270 milliosmolar/liter to about 380 milliosmolar/liter.

It is also an object of the present invention to provide the composition as mentioned above, wherein the composition is stable at room temperature for about 3 months to about 12 months.

It is also an object of the present invention to provide the composition as mentioned above, wherein the composition is stable at fridge temperature for about 6 months to about 24 months.

It is also an object of the present invention to provide the composition as mentioned above, wherein the composition is stable at about 40 degrees Celsius temperature for about 2 months to about 6 months.

It is also an object of the present invention to provide the composition as mentioned above, wherein stability of the composition is measured using a technique selected from the group consisting of measuring drop size, light scattering, focused beam reflectance measurement, centrifugation, rheology and any combination thereof.

It is also an object of the present invention to provide the composition as mentioned above, wherein the composition is to be administered in a route selected from a group consisting of: intranasal, transdermal, intravenous, oral, topical, topical and any combination thereof.

It is also an object of the present invention to provide the composition as mentioned above, wherein the composition is for oral administration in a formulation selected from a group of preparations consisting of syrup, drops, solution, suspension, tablet, bolus, troche, capsule and any combination thereof.

It is also an object of the present invention to provide the composition as mentioned above, wherein the composition is for topical administration in a formulation selected from a group of preparations consisting of cream, ointment lotion, foam, transdermal patch and any combination thereof.

It is also an object of the present invention to provide the composition as mentioned above, wherein the composition is to be administered in combination with at least one pharmaceutical agent.

It is also an object of the present invention to provide the composition as mentioned above, wherein the composition is to be administered in combination with at least one nutraceutical agent.

It is also an object of the present invention to provide the composition as mentioned above, wherein the CBD or the derivative thereof interacts with at least one receptor selected from a group consisting of Cannabinoid receptor type 1 (CB1), Cannabinoid receptor type 2 (CB2), and any combination thereof.

It is also an object of the present invention to provide the composition as mentioned above, wherein the THC or the derivative thereof interacts with at least one receptor selected from a group consisting of Cannabinoid receptor type 1 (CB1), Cannabinoid receptor type 2 (CB2), and any combination thereof.

It is also an object of the present invention to provide the composition as mentioned above, wherein the composition additionally comprises inactive ingredients selected from a group consisting of antiadherents, binders, coatings, disintegrants, flavours, colours, lubricants, glidants, sorbents, preservatives, sweeteners, and any combination thereof.

It is also an object of the present invention to provide the composition as mentioned above, wherein the composition is in a sustained release dosage form; the sustained release dosage form is selected from a group consisting of drug polymer conjugates, microencapsulation, controlled-release tablet coating, and any combination thereof.

It is also an object of the present invention to provide the composition as mentioned above, wherein the composition is nonpsychoactive.

It is also an object of the present invention to provide the composition as mentioned above, wherein the composition is administered once, twice, three or four times through the day.

It is also an object of the present invention to provide the composition as mentioned above, wherein the oily fraction is cannabis oil obtained from at least one cannabis plant.

It is also an object of the present invention to provide the composition as mentioned above, wherein the cannabis plant is a CBD rich strain.

It is also an object of the present invention to provide the composition as mentioned above, wherein the CBD rich strain is selected from a group consisting of Avidekel, Fedora 17, ACDC, and any combination thereof.

It is also an object of the present invention to provide the composition as mentioned above, wherein the cannabis plant is a THC rich strain.

It is also an object of the present invention to provide the composition as mentioned above, wherein the THC rich strain is selected from a group consisting of Black Destroyer, Critical Neville Haze, Mataro Blue, LSD OG Kush, Pineapple Chunk, Blue Monster Holk, Y Griega, Satori, Tutankhamon, and any combination thereof.

It is also an object of the present invention to provide the composition as mentioned above, wherein the CBD or derivative thereof is produced by a synthetic route.

It is also an object of the present invention to provide the composition as mentioned above, wherein the THC or derivative thereof is produced by a synthetic route.

It is also an object of the present invention to provide the composition as mentioned above, wherein the composition is formulated for administration of about 5 mg to about 15 mg THC per dosage unit.

It is also an object of the present invention to provide the composition as mentioned above, wherein the composition further comprises an additional lipophilic solvent or suspension carrier.

It is also an object of the present invention to provide the composition as mentioned above, wherein the lipophilic solvent or suspension carrier are selected from a group consisting of medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, vegetable oil, and any combination thereof.

It is also an object of the present invention to provide the composition as mentioned above, further comprising pH adjusting agents, selected from the group consisting of disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and any combination thereof.

It is also an object of the present invention to provide the composition as mentioned above, further comprising osmotic agents, selected from the group consisting of glycerin, glucose and sucrose, sorbitol, sodium phosphate and any combination thereof.

It is also an object of the present invention to provide the composition as mentioned above, further comprising flavoring agents, selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof.

It is also an object of the present invention to provide the composition as mentioned above, further comprising preservatives, selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

It is another object of the present invention to disclose a composition prepared by the steps of combining phospholipids, or derivatives thereof, and an oily fraction, thereby obtaining an oily phase; combining the oily phase with a water phase in an emulsifier, thereby obtaining pre-emulsion; and transferring the pre-emulsion into a microfluidizer, thereby obtaining a cannabis composition in the formulation of an emulsion; wherein the oily fraction contains about 50% cannabinoids.

It is also an object of the present invention to provide the preparation method as mentioned above, wherein the cannabinoids are selected from the group consisting of cannabidiol (CBD) or a derivative thereof, Tetrahydrocannabinol (THC) or a derivative thereof, and any combination thereof.

It is also an object of the present invention to provide the preparation method as mentioned above, wherein final concentration of the THC or a derivative thereof, in the emulsion is about 100 mg/ml.

It is also an object of the present invention to provide the preparation method as mentioned above, wherein a single dose of about 0.15 ml the cannabis emulsion contains a dose of about 15 mg THC.

It is also an object of the present invention to provide the preparation method as mentioned above, wherein the ratio of the oily fraction and the phospholipid is between 8:1 and 17:1.

It is also an object of the present invention to provide the preparation method as mentioned above, additionally comprising steps of providing the composition with oily fraction in the range of about 5% to about 50%.

It is also an object of the present invention to provide the preparation method as mentioned above, additionally comprising steps of providing the composition with oily fraction in the range of about 10% to about 30%.

It is also an object of the present invention to provide the preparation method as mentioned above, additionally comprising steps of selecting the oily fraction from the group consisting of cannabis oil, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and any combination thereof.

It is also an object of the present invention to provide the preparation method as mentioned above, additionally comprising steps of formulating the oily phase to comprise antioxidants in a final range of about 0.01% to about 0.1% w/v, and selecting the antioxidants from the group consisting of ethanol, polyethylene glycol 300, polyethylene glycol 400, propylene glycol, propylene carbonate, N-methyl-2-pyrrolidones, dimethylacetamide, dimethyl sulfoxide, hydroxypropyl-β-cyclodextrins, sulfobutylether-β-cyclodextrin, α-cyclodextrin, HSPC phospholipid, DSPG phospholipid, DMPC phospholipid, DMPG phospholipid, ascorbyl palmitate, butylated hydroxy anisole, butylatedhydroxy anisole, propyl gallate, α-tocopherol, γ-tocopherol and any combination thereof.

It is also an object of the present invention to provide the preparation method as mentioned above, additionally comprising steps of formulating the oily phase to comprise co-surfactants in a final range of about 1% to about 10% w/v, and selecting the co-surfactants from the group consisting of glycerol, sodium stearate, potassium laurate, sodium dodecyl sulfate, sodium sulfosuccinate, polyglycol, fatty acid esters, quaternary ammonium salts, amine hydrochlorides and any combination thereof.

It is also an object of the present invention to provide the preparation method as mentioned above, additionally comprising steps of formulating the oily phase to comprise chelating agents in a final range of about 0.01% to about 0.5% w/v, and selecting the chelating agents from the group consisting of Ethylenediaminetetraacetic acid (EDTA), phosphoric acid, polyphosphates, polysaccharides, citric acid and any combination thereof.

It is also an object of the present invention to provide the preparation method as mentioned above, additionally comprising steps of selecting the phospholipids from the group consisting of phosphatidylcholine, diphosphatidylglycerol, phosphatidylethanolamine, phosphatidylserine, phosphatidylinositol, sphingomyelin, PEG phospholipid and any combination thereof.

It is also an object of the present invention to provide the preparation method as mentioned above, additionally comprising steps of deriving the phospholipids, or derivatives thereof, of naturally-occurring food sources selected from the group consisting of poultry eggs, soya, rapeseed, sunflower, cattle milk, fish eggs and any combination thereof.

It is also an object of the present invention to provide the preparation method as mentioned above, additionally comprising steps of producing the phospholipids, or derivative thereof, by a synthetic route.

It is also an object of the present invention to provide the preparation method as mentioned above, wherein the step (c) uses a microfluidizer having a pressure of at least 25,000 PSI, thereby resulting in an average particle size of the emulsion in the range of about 50 nm to about 400 nm.

It is also an object of the present invention to provide the preparation method as mentioned above, wherein the step (c) uses a microfluidizer having a pressure of at least 28,000 PSI, thereby resulting in an average particle size of the emulsion in the range of about 50 nm to about 100 nm.

It is also an object of the present invention to provide the preparation method as mentioned above, wherein the step (c) results in an average particle size of said emulsion in the range of about 100 nm to about 400 nm.

It is also an object of the present invention to provide the preparation method as mentioned above, additionally comprising the step of sterilizing the cannabis composition.

It is also an object of the present invention to provide the preparation method as mentioned above, additionally comprising steps of adjusting the cannabis composition's pH to be in the range of about 6.5 to about 7.5.

It is also an object of the present invention to provide the preparation method as mentioned above, additionally comprising steps of adjusting the cannabis composition's pH to be in the range of about 7.0 to about 7.5.

It is also an object of the present invention to provide the preparation method as mentioned above, additionally comprising steps of adjusting the cannabis composition's osmolarity to be in the range of about 200 milliosmolar/liter to about 500 milliosmolar/liter.

It is also an object of the present invention to provide the preparation method as mentioned above, additionally comprising steps of adjusting the cannabis composition's osmolarity to be in the range of about 270 milliosmolar/liter to about 380 milliosmolar/liter.

It is also an object of the present invention to provide the preparation method as mentioned above, additionally comprising steps of formulating the composition to be stable at room temperature for about 3 months to about 12 months.

It is also an object of the present invention to provide the preparation method as mentioned above, additionally comprising steps of formulating the composition to be stable at fridge temperature for about 6 months to about 24 months.

It is also an object of the present invention to provide the preparation method as mentioned above, additionally comprising steps of formulating the composition to be stable at 40 degrees Celsius temperature for about 2 months to about 6 months.

It is also an object of the present invention to provide the preparation method as mentioned above, additionally comprising steps of measuring stability of the composition using a technique selected from the group consisting of measuring drop size, light scattering, focused beam reflectance measurement, centrifugation, rheology and any combination thereof.

It is also an object of the present invention to provide the preparation method as mentioned above, additionally comprising steps of formulating the cannabis composition to be administered in a route selected from a group consisting of: intranasal, transdermal, intravenous, oral, topical, and any combination thereof.

It is also an object of the present invention to provide the preparation method as mentioned above, additionally comprising steps of formulating the cannabis composition to be administered orally in a formulation selected from a group of preparations consisting of syrup, drops, solution, suspension, tablet, bolus, troche, capsule and any combination thereof.

It is also an object of the present invention to provide the preparation method as mentioned above, additionally comprising steps of formulating the cannabis composition to be administered topically in a formulation selected from a group of preparations consisting of cream, ointment lotion, foam, transdermal patch and any combination thereof.

It is also an object of the present invention to provide the preparation method as mentioned above, additionally comprising steps of formulating the cannabis composition with at least one pharmaceutical agent.

It is also an object of the present invention to provide the preparation method as mentioned above, additionally comprising steps of formulating the cannabis composition with at least one nutraceutical agent.

It is also an object of the present invention to provide the preparation method as mentioned above, additionally comprising steps of formulating the cannabis composition with an inactive ingredient selected from a group consisting of antiadherents, binders, coatings, disintegrants, flavours, colours, lubricants, glidants, sorbents, preservatives, sweeteners, and any combination thereof.

It is also an object of the present invention to provide the preparation method as mentioned above, additionally comprising steps of formulating the cannabis composition in a sustained release dosage form; the sustained release dosage form is selected from a group consisting of drug polymer conjugates, microencapsulation, controlled-release tablet coating, and any combination thereof.

It is also an object of the present invention to provide the preparation method as mentioned above, wherein the oily fraction is cannabis oil obtained from at least one cannabis plant.

It is also an object of the present invention to provide the preparation method as mentioned above, wherein the cannabis plant is a CBD rich strain.

It is also an object of the present invention to provide the preparation method as mentioned above, wherein the CBD rich strain is selected from a group consisting of Avidekel, Fedora 17, ACDC, and any combination thereof.

It is also an object of the present invention to provide the preparation method as mentioned above, wherein the cannabis plant is a THC rich strain.

It is also an object of the present invention to provide the preparation method as mentioned above, wherein the THC rich strain is selected from a group consisting of Black Destroyer, Critical Neville Haze, Mataro Blue, LSD OG Kush, Pineapple Chunk, Blue Monster Holk, Y Griega, Satori, Tutankhamon, and any combination thereof.

It is also an object of the present invention to provide the preparation method as mentioned above, wherein the CBD or derivative thereof is produced by a synthetic route.

It is also an object of the present invention to provide the preparation method as mentioned above, wherein the THC or derivative thereof is produced by a synthetic route.

It is also an object of the present invention to provide the preparation method as mentioned above, additionally comprising steps of formulating the cannabis composition for administration of about 5 mg to about 15 mg THC per dosage unit.

It is also an object of the present invention to provide the preparation method as mentioned above, additionally comprising steps of formulating the cannabis composition to contain an additional lipophilic solvent or suspension carrier.

It is also an object of the present invention to provide the preparation method as mentioned above, additionally comprising steps of formulating the cannabis composition to further contain pH adjusting agents, and selecting the pH adjusting agents from the group consisting of disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and any combination thereof.

It is also an object of the present invention to provide the preparation method as mentioned above, additionally comprising steps of formulating the cannabis composition to further contain osmotic agents, and selecting the osmotic agents from the group consisting of glycerin, glucose and sucrose, sorbitol, sodium phosphate and any combination thereof.

It is also an object of the present invention to provide the preparation method as mentioned above, additionally comprising steps of formulating the cannabis composition to further contain flavoring agents, and selecting the flavoring agents from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof.

It is also an object of the present invention to provide the preparation method as mentioned above, additionally comprising steps of formulating the cannabis composition to further contain preservatives, and selecting the preservatives from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

It is another object of the present invention to disclose a method of treating or preventing a medical condition in a subject; the method comprising administrating to the subject a therapeuatically effective amount of a composition comprising phospholipids, or derivatives thereof, and an oily fraction, the composition is formulated as an emulsion, wherein the oily fraction contains about 50% cannabinoids.

It is also an object of the present invention to disclose the aforementioned method, wherein the cannabinoids are selected from the group consisting of cannabidiol (CBD) or a derivative thereof, Tetrahydrocannabinol (THC) or a derivative thereof, and any combination thereof.

It is also an object of the present invention to disclose the aforementioned method, additionally comprising steps of administering the composition with a final concentration of the THC or a derivative thereof, in the emulsion of about 100 mg/ml.

It is also an object of the present invention to disclose the aforementioned method, additionally comprising steps of administering the composition in a single dose of about 0.15 ml the cannabis emulsion containing a dose of about 15 mg THC.

It is also an object of the present invention to disclose the aforementioned method, additionally comprising steps of providing the composition with oily fraction is in the range of about 5% to about 50%.

It is also an object of the present invention to disclose the aforementioned method, additionally comprising steps of providing the composition with oily fraction is in the range of about 10% to about 30%.

It is also an object of the present invention to disclose the aforementioned method, additionally comprising the step of providing the composition wherein the oily fraction is selected from the group consisting of cannabis oil, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and any combination thereof.

It is also an object of the present invention to disclose the aforementioned method, additionally comprising steps of providing the composition with antioxidants in the range of about 0.01% to about 0.1% w/v, wherein the antioxidants are selected from the group consisting of ethanol, polyethylene glycol 300, polyethylene glycol 400, propylene glycol, propylene carbonate, N-methyl-2-pyrrolidones, dimethylacetamide, dimethyl sulfoxide, hydroxypropyl-β-cyclodextrins, sulfobutylether-β-cyclodextrin, α-cyclodextrin, HSPC phospholipid, DSPG phospholipid, DMPC phospholipid, DMPG phospholipid, ascorbyl palmitate, butylated hydroxy anisole, butylatedhydroxy anisole, propyl gallate, α-tocopherol, γ-tocopherol and any combination thereof.

It is also an object of the present invention to disclose the aforementioned method, additionally comprising steps of providing the composition with co-surfactants in the range of about 1% to about 10% w/v, wherein the co-surfactants are selected from the group consisting of glycerol, sodium stearate, potassium laurate, sodium dodecyl sulfate, sodium sulfosuccinate, polyglycol, fatty acid esters, quaternary ammonium salts, amine hydrochlorides and any combination thereof.

It is also an object of the present invention to disclose the aforementioned method, additionally comprising steps of providing the composition with chelating agents in the range of about 0.01% to about 0.5% w/v, wherein the chelating agents are selected from the group consisting of Ethylenediaminetetraacetic acid (EDTA), phosphoric acid, polyphosphates, polysaccharides, citric acid and any combination thereof.

It is also an object of the present invention to disclose the aforementioned method, additionally comprising steps of selecting the phospholipids from the group consisting of phosphatidylcholine, phosphatidylglycerol, phosphatidylethanolamine, phosphatidylserine, phosphatidylinositol, sphingomyelin, PEG phospholipid and any combination thereof.

It is also an object of the present invention to disclose the aforementioned method, wherein the phospholipids, or derivatives thereof, are derived of naturally-occurring food sources selected from the group consisting of poultry eggs, soya, rapeseed, sunflower, cattle milk, fish eggs and any combination thereof.

It is also an object of the present invention to disclose the aforementioned method, wherein the phospholipids, or derivative thereof, are derived by a synthetic route.

It is also an object of the present invention to disclose the aforementioned method, wherein the average particle size of the emulsion is in the range of about 50 nm to about 400 nm.

It is also an object of the present invention to disclose the aforementioned method, wherein particle size of the emulsion is in the range of about 75 nm to about 150 nm.

It is also an object of the present invention to disclose the aforementioned method, wherein particle size of the emulsion is in the range of about 100 nm to about 400 nm.

It is also an object of the present invention to disclose the aforementioned method, wherein the pH of the composition is in the range of about 6.5 to about 7.5.

It is also an object of the present invention to disclose the aforementioned method, wherein the pH of the composition is in the range of about 7.0 to about 7.5.

It is also an object of the present invention to disclose the aforementioned method, wherein the osmolarity of the composition is in the range of about 200 milliosmolar to about 500 milliosmolar.

It is also an object of the present invention to disclose the aforementioned method, wherein the osmolarity of the composition is in the range of about 270 milliosmolar to about 380 milliosmolar.

It is also an object of the present invention to disclose the aforementioned method, additionally comprising steps of administering the composition in a route selected from a group consisting of: intranasal, transdermal, intravenous, oral, topical, and any combination thereof.

It is also an object of the present invention to disclose the aforementioned method, additionally comprising steps of administering the composition orally in a formulation selected from a group of preparations consisting of syrup, drops, solution, suspension, tablet, bolus, troche, capsule and any combination thereof.

It is also an object of the present invention to disclose the aforementioned method, additionally comprising steps of administering the composition topically in a formulation selected from a group of preparations consisting of cream, ointment, lotion, foam, transdermal patch and any combination thereof.

It is also an object of the present invention to disclose the aforementioned method, additionally comprising steps of administering the composition in combination with at least one pharmaceutical agent.

It is also an object of the present invention to disclose the aforementioned method, additionally comprising steps of administering the composition in combination with at least one nutraceutical agent.

It is also an object of the present invention to disclose the aforementioned method, additionally comprising steps of administering the composition over a time period of about 1 day to about 6 months.

It is also an object of the present invention to disclose the aforementioned method, additionally comprising steps of administering the composition once, twice, three or four times through the day.

It is also an object of the present invention to disclose the aforementioned method, wherein the administration does not cause a psychoactive effect.

It is also an object of the present invention to disclose the aforementioned method, additionally comprising steps of providing the composition with pH adjusting agents, selected from the group consisting of disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and any combination thereof.

It is also an object of the present invention to disclose the aforementioned method, additionally comprising steps of providing the composition with osmotic agents, selected from the group consisting of glycerin, glucose and sucrose, sorbitol, sodium phosphate and any combination thereof.

It is also an object of the present invention to disclose the aforementioned method, additionally comprising steps of providing the composition with flavoring agents, selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof.

It is lastly an object of the present invention to disclose the aforementioned method, additionally comprising steps of providing the composition with preservatives, selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

### BRIEF DESCRIPTION OF THE PREFERRED EMBODIMENTS

The novel features believed to be characteristics of the invention are set forth in the appended claims. The invention itself, however, as well as the preferred mode of use, further objects and advantages thereof, will best be understood by reference to the following detailed description of illustrative embodiment when read in conjunction with the accompanying drawings, wherein:

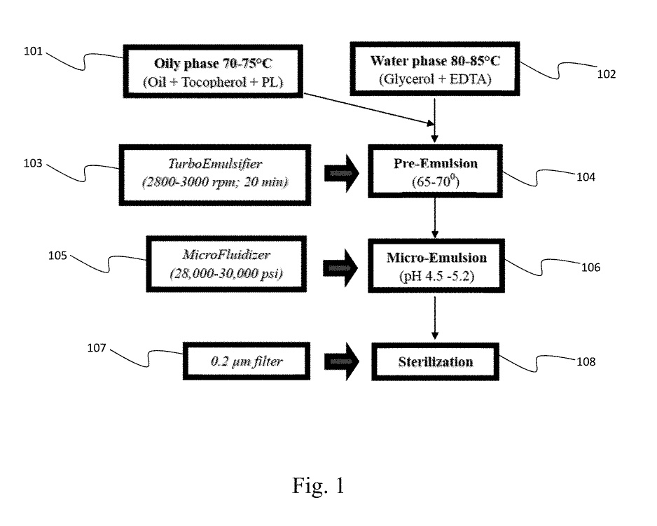

FIG. 1 schematically presents the method of manufacturing of the composition provided in the present invention, in accordance with a preferred embodiment of the present invention;

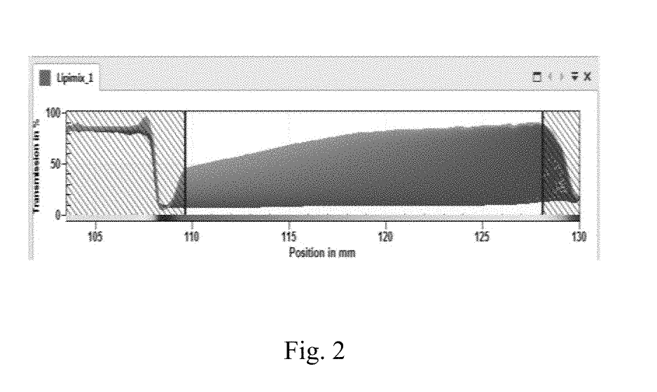

FIGS. 2-6 present graphs of the Instability indexes of emulsions samples, in accordance with a preferred embodiment of the present invention;

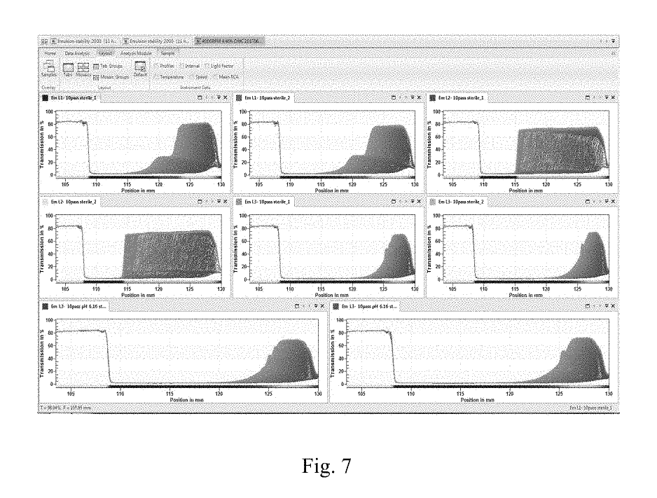

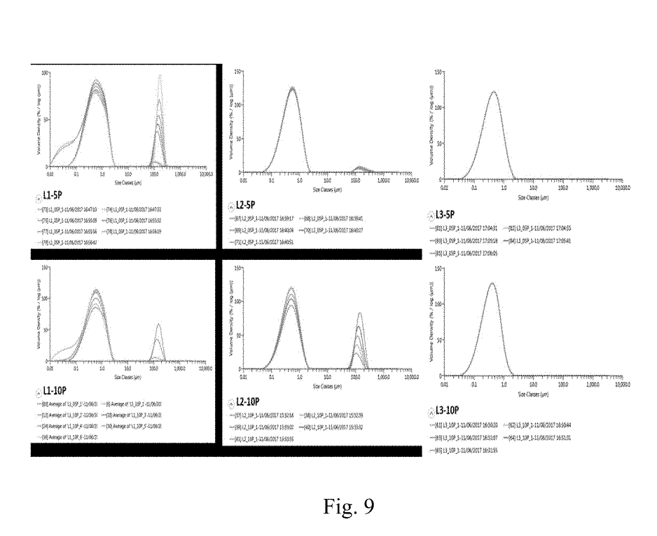

FIGS. 7 and 9 present graphs of the Mean droplets diameters of emulsion samples, in accordance with a preferred embodiment of the present invention;

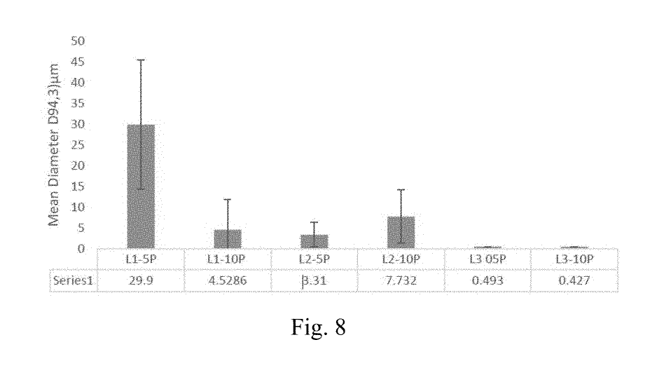

FIGS. 8 and 10-11 present graphs of the Droplets size distributions of multiple measurements of emulsions samples, in accordance with a preferred embodiment of the present invention.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

The essence of the present invention is to provide a composition comprising oil and phospholipids, formulated as an emulsion and useful for administering to a human patient, having used an oily fraction containing about 50% cannabinoids. Such cannabinoids may be either cannabidiol (CBD) and/or Tetrahydrocannabinol (THC) or an extract thereof. More specifically the present invention recites a composition comprising cannabinoids enriched emulsions for either topical or oral use, and most preferably for nasal administration.

The term “about” refers hereinafter to a range of ±25% of the defined amount or measure or value.

The term “cannabidiol (CBD)” refers hereinafter to one of at least 85 active cannabinoids identified in cannabis. Cannabidiol is a major phytocannabinoid, accounting for up to 40% of the plant's extract. CBD is considered to have a wider scope of medical applications than Tetrahydrocannabinol (THC). Cannabidiol has a very low affinity for CB1 and CB2 receptors but acts as an indirect antagonist of their agonists. CBD may potentiate THC's effects by increasing CB1 receptor density or through another CB1-related mechanism. It is also an inverse agonist of CB2 receptors. CBD possesses antiproliferative, pro-apoptotic effects and inhibits cancer cell migration, adhesion and invasion.

The term “Tetrahydrocannabinol (THC)” refers hereinafter to the principal psychoactive constituent (or cannabinoid) of the cannabis plant. THC has a partial agonist activity at the cannabinoid receptor CB1, and the CB2 receptor. THC may refer to delta-9-tetrahydrocannabinol, delta-6-tetrahydrocannabinol and delta-1-tetrahydrocannabinol.

The term “THC rich cannabis strain” refers hereinafter to a cannabis strain having 20% or more THC. More specifically the term relates but is not limited to the following strains: Black Destroyer, Critical Neville Haze, Mataro Blue, LSD OG Kush, Pineapple Chunk, Blue Monster Holk, Y Griega, Satori, Tutankhamon.

The term “CBD rich cannabis strain” refers hereinafter to a cannabis strain having 1% or more CBD. More specifically the term relates but is not limited to the following strains: Avidekel, Fedora 17, ACDC.

The term “Avidekel” refers hereinafter to a cannabis strain comprising 15.8% CBD and less than 1% THC which may be found in patent application US 2014/0259228.

The term “Fedora 17” refers hereinafter to a cannabis strain having a cannabionoid profile consistently around 1% CBD with THC less than 0.1%.

The term “ACDC” refers hereinafter to a cannabis strain having about 19% CBD and a THC/CBD ration of about 1:20.

The term “cannabinoid receptor” refers hereinafter to a class of cell membrane receptors under the G protein-coupled receptor superfamily. There are currently two known subtypes of cannabinoid receptors, termed CB1 and CB2. The CB1 receptor is expressed mainly in the brain, but also in the lungs, liver and kidneys. The CB2 receptor is expressed mainly in the immune system and in hematopoietic cells.

The term “Cannabinoid receptor type 1 (CB1)” refers hereinafter to a G protein-coupled cannabinoid receptor located primarily in the central and peripheral nervous system. It is activated by the endocannabinoid neurotransmitters anandamide and 2-arachidonoyl glyceride (2-AG); by plant cannabinoids, such as the compound THC, an active ingredient of the psychoactive drug cannabis; and by synthetic analogues of THC.

The term “Cannabinoid receptor type 2 (CB2)” refers hereinafter to a G protein-coupled receptor from the cannabinoid receptor family that in humans is encoded by the CNR2 gene. It is closely related to the cannabinoid receptor type 1, which is largely responsible for the efficacy of endocannabinoid-mediated presynaptic-inhibition, the psychoactive properties of Tetrahydrocannabinol, the active agent in marijuana, and other phytocannabinoids (natural cannabinoids). The principal endogenous ligand for the CB2 receptor is 2-arachidonoylglycerol (2-AG).

The term “nonpsychoactive” refers hereinafter not affecting the mind or mental processes.

The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids.

The term “sustained release dosage form” refers hereinafter to the release of a drug at a predetermined rate in order to maintain a constant drug concentration for a specific period of time with minimum side effects. This can be achieved through a variety of formulations, including liposomes and drug-polymer conjugates. Sustained release's definition is more akin to a “controlled release” rather than “sustained”.

The term “particle size” refers hereinafter to oil in water droplet diameter, or water in oil droplet diameter, in an emulsion.

The term “room temperature” refers hereinafter to about 20 to about 25 celcius degrees.

The term “fridge temperature” refers hereinafter to about 2 to about 8 celcius degrees.

According to one embodiment, the present invention provides a composition comprising Tetrahydrocannabinol (THC), Cannabidiol (CBD) or derivative(s) and combinations thereof for use in relieving migraine attack of a patient.

The term “stable” refers hereinafter to the stability of the emulsion as disclosed in the present invention, and specifically refers to the ability of the emulsion to resist change in its properties over time. Instability may be manifested in any of the following: flocculation, creaming, coalescence and Ostwald ripening. Determination whether an emulsion has lost its stability may be carried out in any of the following techniques: measurement of particle size, light scattering, focused beam reflectance measurement, centrifugation or rheology.

It is an object of the present invention to disclose a cannabis emulsion composition comprising phospholipids, or derivatives thereof, and an oily fraction, wherein the oily fraction is enriched and contains about 50% cannabinoids. Such cannabinoids may be selected from the group consisting of cannabidiol (CBD) or a derivative thereof, Tetrahydrocannabinol (THC) or a derivative thereof, and any combination thereof.

Several factors may account for the low oral bioavailability of 4-20% (as compared to intravenous drug administration), including variable absorption, degradation of drug in the stomach, and extensive first-pass metabolism to metabolites in the liver. Therefore there is a need to develop a formulation with improved bioavailability, rapid absorption, and overcoming the first pass hepatic metabolism.

The micro emulsion of the present invention comprises small-size particles, produced without the using of surfactants, thus avoiding the pungent, irritating side-effect of said surfactants.

In preferred embodiments, the micro emulsion composition of the present invention is suitable for variety routes of administration, bypassing the hepatic first pass metabolism, and enabling rapid absorption ad high bioavailability.

In preferred embodiments, the micro emulsion composition is provided with a lower viscosity.

In preferred embodiments, the micro emulsion composition is provided with a pH range of about 5.5-7.5, further improving its safety level.

In preferred embodiments, the micro-emulsion composition is useful for transferring variety of molecules specifically fatty molecules, without sediments or any solid particles.

In preferred embodiments, the micro emulsion composition of the present invention is set for predetermined dose of said cannabinoids or a mixture of at least two cannabinoids.

In preferred embodiments, the micro emulsion composition of the present invention having a predefined dosage form and predefined THC/CBD ration, when administered absorbed by enriched mucosa and bypass liver route metabolism. According to other embodiments, the emulsion composition of the present invention is characterized by a desirable viscosity properties, specifically, lower viscosity as compared to the cannabis oil which is the active ingredient of the disclosed formulation. This property provides higher bioavailability and penetration into mucosal membranes and tissues such as in the nasal cavity.

In preferred embodiments, the final concentration of THC, or a derivative thereof, in the emulsion formulation is about 100 mg/ml. Thus, a single dose of about 0.15 ml of the cannabis emulsion, which is particularly suitable for nasal administration, contains a dose of about 15 mg THC. The use of enriched oily fraction having about 50% cannabinoids enables administration of effective therapeutic amounts of cannabinoids in a much smaller volume of a dose, thereby permitting the composition to be administered in various pathways, which have not been available up until the present invention.

It is within the scope to provide the composition as defined in any of the above wherein the ratio of the oily fraction and the phospholipid is between 8:1 and 17:1.

It is further within the scope to provide the composition as defined in any of the above wherein the oily fraction is in the range of about 5% to about 50%, or in the range of about 10% to about 30%.

It is further within the scope to provide the composition as defined in any of the above, wherein the oily fraction may be selected from the group consisting of cannabis oil (hemp oil), borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylic/capric glycerides, and any combination thereof.

It is further within the scope to provide the composition as defined in any of the above, further comprising further comprising antioxidants in the range of about 0.01% to about 0.1% w/v, and wherein the antioxidants may be selected from the group consisting of ethanol, polyethylene glycol 300, polyethylene glycol 400, propylene glycol, propylene carbonate, N-methyl-2-pyrrolidones, dimethylacetamide, dimethyl sulfoxide, hydroxypropyl-β-cyclodextrins, sulfobutylether-β-cyclodextrin, α-cyclodextrin, HSPC phospholipid, DSPG phospholipid, DMPC phospholipid, DMPG phospholipid, ascorbyl palmitate, butylated hydroxy anisole, butylatedhydroxy anisole, propyl gallate, α-tocopherol, γ-tocopherol and any combination thereof.

It is further within the scope to provide the composition as defined in any of the above, further comprising co-surfactants in the range of about 1% to about 10% w/v, and these co-surfactants may be selected from the group consisting of glycerol, sodium stearate, potassium laurate, sodium dodecyl sulfate, sodium sulfosuccinate, polyglycol, fatty acid esters, quaternary ammonium salts, amine hydrochlorides and any combination thereof.

It is further within the scope to provide the composition as defined in any of the above, further comprising chelating agents in the range of about 0.01% to about 0.5% w/v, and the chelating agents may be selected from the group consisting of Ethylenediaminetetraacetic acid (EDTA), phosphoric acid, polyphosphates, polysaccharides, citric acid and any combination thereof.

It is further within the scope to provide the composition as defined in any of the above, wherein the phospholipids are selected from the group consisting of phosphatidylcholine, phosphatidylinositol, diphosphatidylglycerol, phosphatidylethanolamine, phosphatidylserine, sphingomyelin, PEG phospholipid and any combination thereof. The phospholipids may also be derived of naturally-occurring food sources such as, but not limited to, poultry eggs, soya, rapeseed, sunflower, cattle milk, fish eggs and any combination thereof. In other embodiments, the phospholipids, or derivative thereof, are produced by a synthetic route.

The composition as provided in the present invention may be manufactured to provide emulsion particle sizes in the range of about 50 nm to about 200 nm, or in the range of about 75 nm to about 150 nm. Such particularly small particle size is achieved through using a microfluidizer having a pressure of about 25,000 PSI to about 35,000 PSI.

It is further within the scope to provide the composition as defined in any of the above wherein the composition's pH is in the range of about 6.5 to about 7.5.

It is further within the scope to provide the composition as defined in any of the above wherein the composition's pH is in the range of about 5.5 to about 7.5.

It is further within the scope to provide the composition as defined in any of the above wherein the composition's osmolarity is in the range of about 200 milliosmolar/liter to about 500 milliosmolar/liter.

It is further within the scope to provide the composition as defined in any of the above wherein the composition is stable at room temperature for about 3 months to about 12 months, or wherein the composition is stable at fridge temperature for about 6 months to about 24 months. Stability of the composition may be measured using a technique such as, but not limited to, particle size, light scattering, focused beam reflectance measurement, centrifugation, rheology and any combination thereof.

It is further within the scope to provide the composition as defined in any of the above, wherein the composition is for oral administration in a formulation selected from a group of preparations consisting of syrup, drops, solution, suspension, tablet, bolus, troche, capsule and any combination thereof.

It is further within the scope to provide the composition as defined in any of the above, wherein the composition is for topical administration in a formulation selected from a group of preparations consisting of cream, ointment lotion, foam, transdermal patch and any combination thereof.

It is further within the scope to provide the composition as defined in any of the above, wherein the composition is to be administered in combination with at least one pharmaceutical agent. Such a pharmaceutical agent may be any medication having a clinical effect on a human patient, and especially preferred are pharmaceutical compositions directed towards medical conditions which may also benefit from administration of cannabinoids, such as in pain management, nausea, appetite stimulation and the like.

It is further within the scope to provide the composition as defined in any of the above, wherein the composition is to be administered in combination with at least one nutraceutical agent, such as any plant-derived nutrients, synthetically derived nutrients, dietary supplements or herbal products.

It is further within the scope to provide the composition as defined in any of the above, wherein the CBD or the derivative thereof interacts with at least one receptor selected from a group consisting of Cannabinoid receptor type 1 (CB1), Cannabinoid receptor type 2 (CB2), and any combination thereof, and wherein the THC or the derivative thereof interacts with at least one receptor selected from a group consisting of Cannabinoid receptor type 1 (CB1), Cannabinoid receptor type 2 (CB2), and any combination thereof.

It is further within the scope to provide the composition as defined in any of the above, wherein the composition additionally comprises inactive ingredients selected from a group consisting of antiadherents, binders, coatings, disintegrants, flavours, colours, lubricants, glidants, sorbents, preservatives, sweeteners, and any combination thereof.

It is further within the scope to provide the composition as defined in any of the above, wherein the composition is in a sustained release dosage form, such as, but not limited to, drug polymer conjugates, microencapsulation, controlled-release tablet coating, and any combination thereof.

It is further within the scope to provide the composition as defined in any of the above, wherein the composition is nonpsychoactive and does not exhibit any psycho-effect on the user.

It is further within the scope to provide the composition as defined in any of the above, wherein the composition is administered once, twice, three or four times through the day.

In various embodiments, the cannabis oil is obtained from at least one cannabis plant. This plant may be either a CBD rich strain, such as Avidekel, Fedora 17, ACDC, or it may be a THC rich strain, such as Black Destroyer, Critical Neville Haze, Mataro Blue, LSD OG Kush, Pineapple Chunk, Blue Monster Holk, Y Griega, Satori, Tutankhamon.

In other embodiments, CBD or derivative thereof, or THC or derivative thereof, may be produced by a synthetic route.

It is further within the scope to provide the composition as defined in any of the above, wherein the composition is formulated for administration of about 5 mg to about 15 mg THC per dosage unit. In other embodiments, the composition is formulated for administration of about 10 mg THC per dosage unit. In yet other embodiments, the composition is formulated for administration of about 1 mg to about 20 mg THC per dosage unit.

It is further within the scope to provide the composition as defined in any of the above, further comprising an additional lipophilic solvent or suspension carrier, which may be in a non-limiting example, medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, vegetable oil, and any combination thereof. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

It is further within the scope to provide the composition as defined in any of the above, further comprising pH adjusting agents, which may be selected from the group consisting of disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and any combination thereof.

It is further within the scope to provide the composition as defined in any of the above, further comprising osmotic agents, which may be selected from the group consisting of glycerin, glucose and sucrose, sorbitol, sodium phosphate and any combination thereof.

It is further within the scope to provide the composition as defined in any of the above, further comprising flavoring agents, which may be selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof.

It is further within the scope to provide the composition as defined in any of the above, further comprising preservatives, which may be selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

It is further within the scope to provide the composition as defined in any of the above, wherein the THC or a derivative thereof is selected from the group consisting of THC, THCV, THCA, THCVA and any combination thereof.

It is further within the scope to provide the composition as defined in any of the above, wherein the THC or a derivative thereof is selected from the group consisting of natural THC or a derivative thereof produced in the body of humans and animals, THC or a derivative thereof extracted from plants, synthetic THC or a derivative thereof, and any combination thereof.

It is further within the scope to provide the composition as defined in any of the above, wherein the THC or a derivative thereof is extracted from cannabis; the cannabis is selected from a group consisting of: Cannabis sativa, Cannabis indica, Cannabis ruderalis, and any combination thereof.

It is further within the scope to provide the composition as defined in any of the above, wherein the composition further comprises at least one additional cannabinoid or a derivative thereof.

It is further within the scope to provide the composition as defined in any of the above, wherein the composition provides a synergistic effect with respect to relieving a medical condition as compared to the effect provided by THC or a derivative thereof or by CBD or a derivative thereof administered separately.

It is further within the scope to provide the composition as defined in any of the above, wherein the cannabidiol (CBD) or a derivative thereof is selected from the group consisting of CBD, CBDV, CBDA and any combination thereof.

It is further within the scope to provide the composition as defined in any of the above, wherein the CBD or a derivative thereof is selected from the group consisting of natural CBD or a derivative thereof produced in the body of humans and animals, CBD or a derivative thereof extracted from plants, synthetic CBD or a derivative thereof, and any combination thereof.

It is further within the scope to provide the composition as defined in any of the above, wherein the CBD or a derivative thereof is extracted from cannabis; the cannabis is selected from a group consisting of: Cannabis sativa, Cannabis indica, Cannabis ruderalis, and any combination thereof.

It is further within the scope to provide the composition as defined in any of the above, wherein the composition is administered in a manner selected from a group consisting of: intranasal, transdermal, intravenous, oral, and any combination thereof.

It is further within the scope to provide the composition as defined in any of the above, wherein the composition is formulated in a dosage form selected from a group consisting of liquid, solid, gas, oral, pill, tablet, capsule, buccal, sub-lingual, orally-disintegrating, thin film, liquid solution, suspension, powder or liquid or solid crystals, pastes, inhalational, aerosol, inhaler, nebulizer, smoking, vaporizer, parenteral, intradermal, intramuscular, intraosseous, intraperitoneal, intravenous, subcutaneous, topical, cream, gel, liniment or balm, lotion, ointment, drops, skin patch, vaginal, suppository, pessary, rectal and any combination thereof.

It is further within the scope to provide the composition as defined in any of the above, wherein the composition additionally comprises at least one carrier or excipient selected from a group consisting of diluents, antiadherents, binders, coatings, disintegrants, surfactants, dissolving agents, solubilising agents, bioadhesive agents, polysaccharides, polymers, copolymers, fast dissolving tablet (FDT) type excipient, bioavailability enhancing agent, Thin Film type excipient, PharmFilm type excipient, mucoadhesive type excipient, acidifying agents, probiotic agents, protective agents, antioxidants, effervescent excipient, dispersing agents flavours, colours, lubricants, glidants, sorbents, preservatives, sweeteners, and any combination thereof.

It is further within the scope to provide the composition as defined in any of the above, wherein the composition is in a sustained release dosage form or in an immediate release dosage form.

It is further within the scope to provide the composition as defined in any of the above, wherein the sustained release dosage form is selected from a group consisting of drug polymer conjugates, microencapsulation, controlled-release tablet coating, and any combination thereof.

Reference is now made to FIG. 1, illustrating the method of manufacturing provided by the present invention, resulting in a composition comprising of phospholipids, or derivatives thereof, and cannabinoids enriched oily fraction, having about 50% cannabinoids, which may be CBD or THC or both. The first step 101 of the manufacturing protocol is by creating the oily phase, comprising of the oily fraction and phospholipids, and preferably an anti-oxidant such as tocopherol. The oily phase is then combined with the water phase 102, which preferably additionally contains glycerol and EDTA. Using a TurboEmulsifier 103, the oily phase and the water phase are combined to provide pre-emulsion 104. The pre-emulsion is passed through the MicroFluidizer 105 which is conducted under extremely high pressure of 28,000-30,000 PSI, and could range from 25,000 to 35,000 PSI, to create a micro-emulsion 106, containing particle size having a range of about 75 nm to about 150 nm diameter, or the range of about 50 nm to about 200 nm. The microemulsion is then preferably passed through a 0.2 μm filter 107, resulting in its sterilization 108. According to some embodiments of the present invention, the micro-emulsion contains Phospholipids and an oily phase. The non-polar molecules are kept inside the Phospholipid structure, in the micro-emulsion.

In order to understand the invention and to see how it may be implemented in practice, a plurality of preferred embodiments will now be described, by way of non-limiting example only, with reference to the following examples.

### Example 1

Proposed Emulsion Composition:

| | | |
|---|---|---|
| Cannabis oil | 20 | g |
| Egg phospholipid | 1.2 | g |
| Glycerol | 2.25 | g |
| Tocopherol | 0.02 | g |
| EDTA | 0.05 | g |
| Purified water | up to 100 | ml |

Proposed Emulsion Specifications:

| | |
|---|---|
| Particle size | 100-200 nm |
| pH | 7.0-7.5 |
| Osmolarity | 270-380 milliosmolar |
| Stability | 6-24 months |
| Sterile | |

Proposed Experimental Design:
Step 1: Small volume preparation
   Equipment: A device for a minimal volume of 10 ml emulsion
Step 2: Large volume preparation
   Equipment: A device for a minimal volume of 50 ml emulsion

### Example 2

Protocol for Preparation of Cannabis Emulsion

Emulsion Composition:
Cannabis oil compositions containing THC and CBD in predetermined ratios
Surfactant: phospholipids and/or tween 80 and/or others.
Glycerol
Antioxidants: Tocopherol and/or EDTA and/or others.
Purified water

Manufacturing Flow Chart:

Pre-Emulsion:

Generating a Pre-Emulsion in a TurboEmulsifier (65-700); containing Oily phase at 70-75° C. and Water phase at 80-85° C.

Micro-Emulsion:

The resulting Pre-Emulsion is then passed in a MicroFluidizer in order to create a Micro-Emulsion;

Sterilization:

The resulting Micro-Emulsion is then passed through a 0.2 μm filter resulting in the Sterilization of the Emulsion.

### Example 3

Quality Control of the Preparation

Analysis Tools:
Stability—determined by centrifugation and shelf stability test
Particle size determination
pH measurement
Osmolarity measurement
Viscosity measurement

Goals of the Quality Control Analysis:

Determination of the optimal parameters for the production of the cannabis emulsion:
TurboEmulsifier speed (rpm) and time of operation (min)
MicroFluidizer pressure (psi) and number of passages
Type and amount of surfactant(s)
Type and amount of antioxidant(s) if required

The final emulsion should comprise the following properties:
Particle size 50-400 nm, preferably between 100-400 nm
pH 6.5-7.5, preferably between 7.0-7.5
Osmolarity 200-500, preferably between 270-380 milliosmolar
Stability 3 months at 40 degrees Celsius

### Example 4

Cannabis Emulsion—Protocol, Results and Conclusions

Table 1 below presents the composition preparation:

TABLE 1

| | Emulsion L3 | |
|---|---|---|
| Oil phase | | |
| Cannabis oil (40/10) (in | 20 | g |
| canola oil) | | |
| Soya phospholipids | 1.2 | g |
| Sodium oleate | 0.03 | g |
| Tocopherol | 0.02 | g |
| Water phase | | |
| Glycerol | 2.25 | g |
| EDTA | 0.05 | g |
| DDW | 80 | g |
| Total volume | 100 | ml |

Preparation Protocol:

1. Oil phase—heating to 65° C. in a water bath for 20 min.

2. Water phase—heating to 75° C. in a water bath for 20 min, mix till the glycerol is dissolved.

3. Adding the water phase to the oil and pre-mix for 2 min at 27,000 RPM (Speed setting no. 4) with manual mixer (D-1 Homogenizer, Miccra, Germany).

4. Preparation of final Emulsion with High-pressure homogenizer (Micro DeBEE) at maximal air pressure of 150 Psi (process pressure of 45 kPsi), at room temperature—5 passes.

5. Measuring pH and correcting to pH in the range of about 7.0-7.4 if needed.

6. Sterilization by autoclave at 121° C. for 20 min.

Analysis Protocol:

7. Performing Stability measurements (LumiSizer at 2000 RPM for 11.5 h, 4° C.) with duplicates.

8. Droplets size measurements (Mastersizer 3000).

Analysis results of Emulsion L3-5 (5 Passes) and L3-10 (10 Passes):

1. pH—6.96

2. Instability Index:

a. L3-5—0.236

b. L3-10—0.386

c. L3-10 sterile—0.442

Lipimix (reference)—0.558

3. Mean particle diameter (μm):

a. L3-5—0.493

b. L3-10—0.427

c. L3-10 sterile—0.414

Lipimix (reference)—0.113

Experimental Protocol and Results

Table 2 below presents the emulsion samples preparation:

TABLE 2

| | Emulsion L1 | | Emulsion L2 | | Emulsion L3 | |
|---|---|---|---|---|---|---|
| Oil phase | | | | | | |
| Cannabis oil (40/10) (in | 20 | g | 20 | g | 20 | g |
| canola oil) | | | | | | |
| Egg phospholipids | 1.2 | g | 2.4 | g | | |
| Soya phospholipids | | | | | 1.2 | g |
| Sodium oleate | | | | | 0.03 | g |
| Tocopherol | 0.02 | g | 0.02 | g | 0.02 | g |
| Water phase | | | | | | |
| Glycerol | 2.25 | g | 2.25 | g | 2.25 | g |
| EDTA | 0.05 | g | 0.05 | g | 0.05 | g |
| DDW | 80 | g | 80 | g | 80 | g |
| Total volume | 100 | ml | 100 | ml | 100 | ml |

Working Protocol:

1. Oil phase—heating to 65° C. in a water bath for 20 min.

2. Water phase—heating to 75 0° C. in a water bath for 20 min, mix till the glycerol is dissolved.

3. Adding the water phase to the oil and pre-mix for 2 minutes at 27,000 RPM (Speed setting no.

4) with manual mixer (D-1 Homogenizer, Miccra, Germany).

4. Preparation of final Emulsion with High-pressure homogenizer (Micro DeBEE) at maximal air pressure of 150 Psi (process pressure of 45 kPsi), at room temperature. Collect sample after 5 passes and 10 passes. Use a new beaker every second pass.

5. Stability measurements (LumiSizer at 2000 RPM for 11.5 h, 4° C.) with duplicates of emulsions: L1-5, L1-10, L2-5, L2-10, L3-5 and L3-10.

6. Measuring droplets size (Mastersizer 3000) of all emulsions: L1-5, L1-10, L2-5, L2-10, L3-5 and L3-10.

7. Divide emulsions L1-10, L2-10, L3-10 to two: a. filter through 0.22 μm (L1-10a, L2-10a, L3-10a). b. measure pH and correct (if needed) to pH between 5.8-6.2 with 0.2 M NaOH solution (L1-10b, L2-10b, L3-10b), (right down NaOH volume) and filter through 0.22 μm. (the filtered emulsion must be sterile).

* Filtration was failed, emulsions L1-10a, L2-10a, L3-10a and L3-10b were sterilized using autoclave at 121° C. for 20 min.

8. Stability measurements (LumiSizer at 2000 RPM for 11.5 h, 4° C.) with duplicates of sterile emulsions: L1-10a, L2-10a, L3-10a, L3-10b.

9. Measuring droplets size (Mastersizer 3000) of sterile emulsions L1-10a, L2-10a, L3-10a, L3-10b.

Results:

Table 3 below presents the volume of HCl 0.5M (10 passes emulsions) which was added to achieve pH 5.8-6.2:

TABLE 3

| | | Vol (μl) |
|---|---|---|
| | | to achieve |
| Emulsion name | pH | pH 5.8-6.2 |
| L1 | 6.25 | 0 |
| L2 | 6.26 | 0 |
| L3 | 6.96 | 50 |

Reference is now made to FIG. 2 which presents a graph of the stability results of the emulsion reference Lipimix after 11:30 hr at 2000 rpm—instability index 0.563.

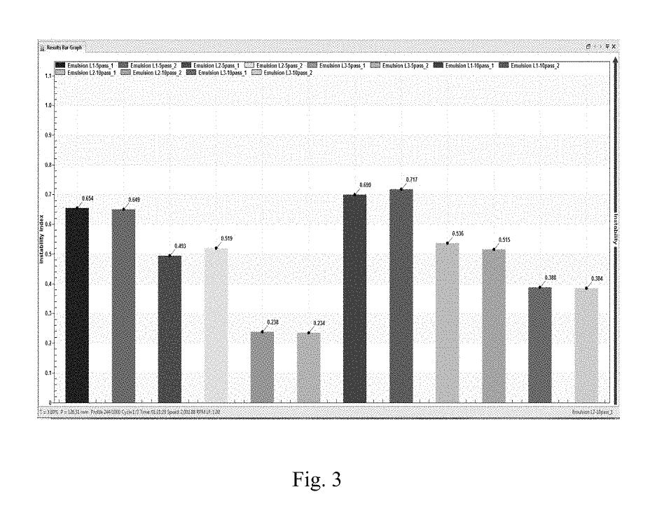

Reference is now made to FIG. 3 which presents a graph of the Instability indexes of emulsion samples L1-5, L1-10, L2-5, L2-10, L3-5, L3-10 at duplicates.

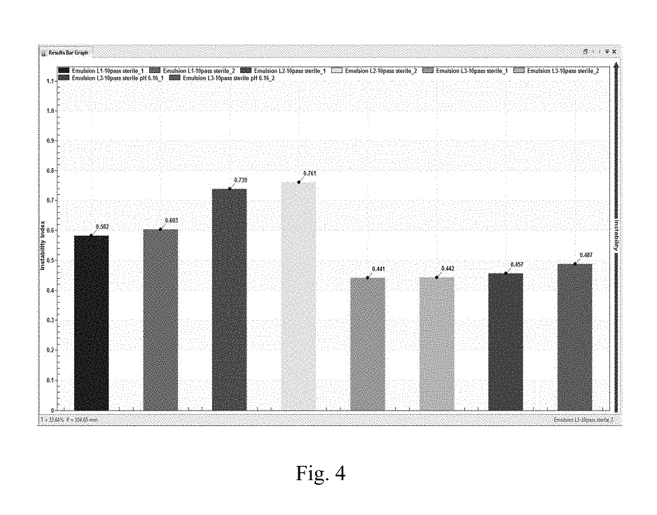

Reference is now made to FIG. 4 which presents a graph of the Instability indexes of sterile emulsions L1-10a, L2-10a, L3-10a and L3-10b at duplicates.

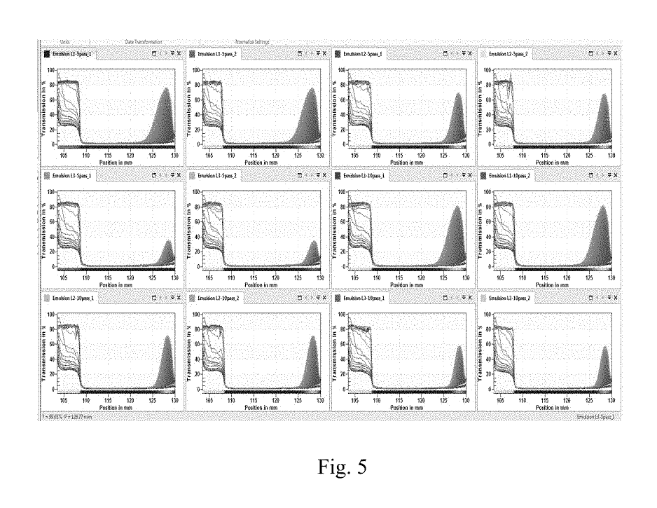

Reference is now made to FIG. 5 which presents LumiSizer images of the emulsion samples after 11 hr at 2000 RPM.

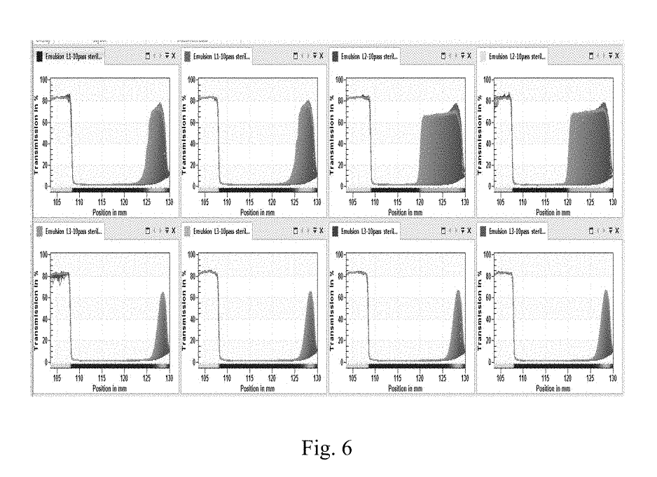

Reference is now made to FIG. 6 which presents images of the sterile emulsion samples after 11 hr at 2000 RPM.

Reference is now made to FIG. 7 which presents images of the sterile emulsion samples after 11 hr at 2000 RPM followed by 4:40 hr at 4000 RPM.

It was further noticed that the emulsion samples that were prepared in a sterile environment (sterilization in an autoclave) after an additional centrifugation for 4:40 hr at 4000 RPM all the samples were with similar stability, however the emulsion sample having the corrected pH (emulsion L3, pH—6.96) was with improved stability.

Reference is now made to FIG. 8 which presents graph of Mean droplets diameters (D[4,3]) of the emulsion samples L1-5, L1-10, L2-5, L2-10, L3-5, L3-10.

Reference is now made to FIG. 9 which presents graphs of the Droplets size distributions of multiple measurements of emulsions L1-5, L1-10, L2-5, L2-10, L3-5, L3-10.

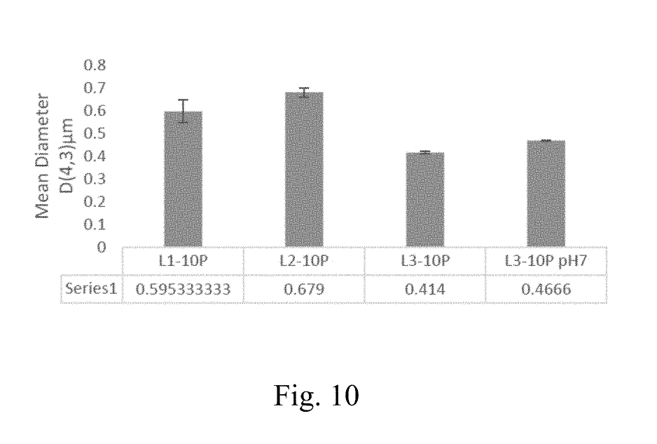

Reference is now made to FIG. 10 which presents graph of the Mean droplets diameters (D[4,3]) of emulsions L1-10a, L2-10a, L3-10a and L3-10b after autoclave sterilization.

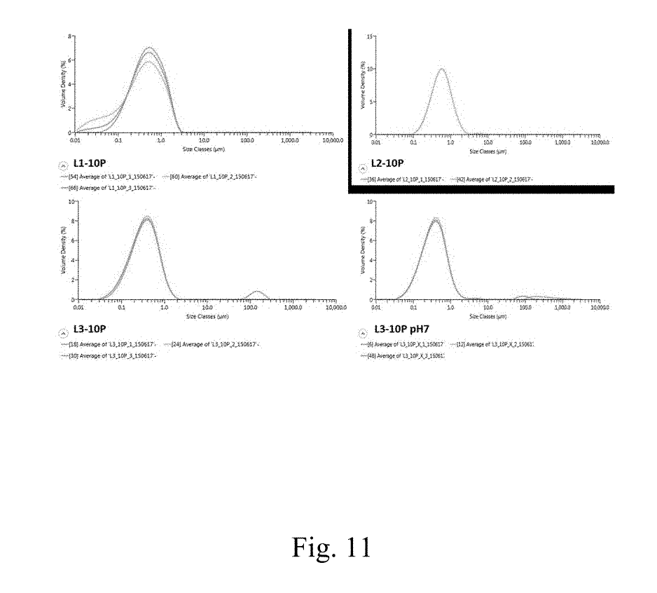

Reference is now made to FIG. 11 which presents graphs of the Droplets size distributions of multiple measurements of emulsions L1-10a, L2-10a, L3-10a and L3-10b.

Table 4 below presents the emulsion samples volume:

TABLE 4

| Emulsion | Sterilized/unsterilized | Volume (ml) |
|---|---|---|
| L1-10a | Sterilized | 20 |
| L2-10a | Sterilized | 30 |
| L3-10a | Sterilized | 35 |
| L1-10a | Sterilized | 35 |
| L1-10a | unsterilized | 30 |
| L2-10a | unsterilized | 30 |
| L1-5 | unsterilized | 5 |
| L2-5 | unsterilized | 5 |
| L3-5 | unsterilized | 5 |

1. A composition comprising phospholipids, or derivatives thereof, and an oily fraction, said composition is formulated as an emulsion;
wherein said oily fraction contains about 50% cannabinoids.

2. The composition of claim 1, wherein said composition comprises small-size particles in a diameter range of about 0.1 μm to 1 μm configured to be absorbed through mucosal membranes.

3. The composition of claim 2, wherein said particles diameter is in the range of about 0.4 μm to 0.45 μm.

4. The composition of claim 1, wherein said composition has instability index in the range of 0.2 to 0.5.

5. The composition of claim 1, wherein said composition has a pH range of about 5.5 to about 7.5.

6. The composition of claim 1, wherein said composition is administered in a manner selected from the group consisting of: intranasal, sublingual, buccal, transdermal, oromucosal, suppository, rectal, intramuscular, inhalational aerosol, transdermal, intravenous, oral, topical and any combination thereof.

7. The composition of claim 1, wherein said composition comprises a variety of molecules specifically fatty molecules, without sediments or any solid particles.

8. The composition according to claim 1, wherein said composition is formulated in a dosage form selected from the group consisting of liquid, solid, gas, oral, pill, tablet, capsule, caplet, buccal, sub-lingual, orally-disintegrating, thin film, liquid solution, suspension, powder or liquid or solid crystals, pastes, inhalational, aerosol, inhaler, nebulizer, smoking, vaporizer, spray, syrup, parenteral, intradermal, intramuscular, intraosseous, intraperitoneal, intravenous, subcutaneous, topical, cream, gel, liniment or balm, lotion, ointment, drops, skin patch, vaginal, suppository, pessary, rectal and any combination thereof.

9. The composition of claim 1, wherein at least one of the following holds true:
a. said composition comprises a mixture of at least two cannabinoids;
b. said composition comprises predetermined ratio of said cannabinoids;
c. said cannabinoid is extracted from cannabis; said cannabis is selected from the group consisting of: Cannabis sativa, Cannabis indica, Cannabis ruderalis, and any combination thereof;
d. said cannabinoid is selected from the group consisting of cannabinoid-type, cannabinoid derivative, cannabis extract or fraction thereof and any combination thereof; and
e. said cannabinoid is selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

10. The composition according to claim 9, wherein at least one of the following holds true:
a. the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof;
b. the cannabidiol (CBD) or a derivative thereof is selected from the group consisting of CBD, CBDV, CBDA and any combination thereof;
c. said THC or a derivative thereof is selected from the group consisting of natural THC or a derivative thereof produced in the body of humans and animals, THC or a derivative thereof extracted from plants, synthetic THC or a derivative thereof, and any combination thereof; and
d. said CBD or a derivative thereof is selected from the group consisting of natural CBD or a derivative thereof produced in the body of humans and animals, CBD or a derivative thereof extracted from plants, synthetic CBD or a derivative thereof, and any combination thereof.

11. The composition of claim 1, wherein at least one of the following holds true:
a. said composition comprising Tetrahydrocannabinol (THC) and Cannabidiol (CBD), or an extract comprising said Tetrahydrocannabinol (THC) and Cannabidiol (CBD), said ratio of said THC:CBD is about 40:10% w/w;
b. said composition is configured to be stable for at least 3 months at up to 40° C.;
c. said composition comprises about 20% cannabis oil;
d. said composition comprises an oily phase and a water phase in a ratio of about 20% to about 80%, respectively;
e. said oily phase comprises about 0.3% of phospholipids; said composition comprises cannabis, surfactant, glycerol, antioxidants and water; and
f. said oily phase comprises cannabis oil, egg phospholipids, soya phospholipids, sodium oleate and tocopherol and wherein said water phase comprises glycerol, EDTA, and water.

12. The composition of claim 11 wherein said cannabis is cannabis oil, said antioxidants are tocopherol or EDTA or any combination thereof; said surfactant is phospholipids or tween 80 or any combination thereof.

13. The composition of claim any one of claims 1 and 9, wherein at least one of the following holds true:
a. said cannabinoids are selected from the group consisting of cannabidiol (CBD) or a derivative thereof, Tetrahydrocannabinol (THC) or a derivative thereof, and any combination thereof;
b. final concentration of said THC or a derivative thereof, in said emulsion is in the range of about 80 to about 100 mg/ml;
c. a single dose of about 0.15 ml of said cannabis emulsion contains a dose of about 12 to about 15 mg THC;
d. the ratio of said oily fraction and said phospholipid is between 8:1 and 17:1;
e. said oily fraction is in the range of about 5% to about 50%; and
f. said oily fraction is in the range of about 10% to about 30%.

14. The composition of claim 1, wherein at least one of the following holds true:
a. said oily fraction is selected from the group consisting of cannabis oil, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, caprylyic/capric glycerides and any combination thereof;
b. said composition further comprising antioxidants in the range of about 0.01% to about 0.1% w/v, and selected from the group consisting of ethanol, polyethylene glycol 300, polyethylene glycol 400, propylene glycol, propylene carbonate, N-methyl-2-pyrrolidones, dimethylacetamide, dimethyl sulfoxide, hydroxypropyl-β-cyclodextrins, sulfobutylether-β-cyclodextrin, α-cyclodextrin, HSPC phospholipid, DSPG phospholipid, DMPC phospholipid, DMPG phospholipid, ascorbyl palmitate, butylated hydroxy anisole, butylatedhydroxy anisole, propyl gallate, α-tocopherol, γ-tocopherol and any combination thereof;
c. said composition further comprising co-surfactants in the range of about 1% to about 10% w/v, and selected from the group consisting of glycerol, sodium stearate, potassium laurate, sodium dodecyl sulfate, sodium sulfosuccinate, polyglycol, fatty acid esters, quaternary ammonium salts, amine hydrochlorides and any combination thereof;
d. said composition further comprising chelating agents in the range of about 0.01% to about 0.5% w/v, and said chelating agents are selected from the group consisting of Ethylenediaminetetraacetic acid (EDTA), phosphoric acid, polyphosphates, polysaccharides, citric acid and any combination thereof;
e. said phospholipids are selected from the group consisting of phosphatidylcholine, diphosphatidylglycerol, phosphatidylethanolamine, phosphatidylserine, phosphatidylinositol, sphingomyelin, PEG phospholipid and any combination thereof;
f. said phospholipids, or derivatives thereof, are derived of naturally-occurring food sources selected from the group consisting of poultry eggs, soya, rapeseed, sunflower, cattle milk, fish eggs and any combination thereof; or said phospholipids, or derivative thereof, are produced by a synthetic route;
g. said composition's osmolarity is in the range of about 200 milliosmolar/liter to about 500 milliosmolar/liter;
h. said composition's osmolarity is in the range of about 270 milliosmolar/liter to about 380 milliosmolar/liter;
i. said composition is stable at room temperature for about 3 months to about 12 months;
j. said composition is stable at fridge temperature for about 6 months to about 24 months;
k. said composition is stable at about 40 degrees Celsius temperature for about 2 months to about 6 months;
l. stability of said composition is measured using a technique selected from the group consisting of measuring drop size, light scattering, focused beam reflectance measurement, centrifugation, rheology and any combination thereof.
m. wherein said composition is to be administered in combination with at least one pharmaceutical agent;
n. said composition is to be administered in combination with at least one nutraceutical agent.
o. said composition additionally comprises inactive ingredients selected from a group consisting of antiadherents, binders, coatings, disintegrants, flavours, colours, lubricants, glidants, sorbents, preservatives, sweeteners, and any combination thereof;
p. said composition is in a sustained release dosage form; said sustained release dosage form is selected from a group consisting of drug polymer conjugates, microencapsulation, controlled-release tablet coating, and any combination thereof;
q. said composition is nonpsychoactive;
r. said composition is administered once, twice, three or four times through the day;
s. said oily fraction is cannabis oil obtained from at least one cannabis plant;
t. said CBD or derivative thereof is produced by a synthetic route; and
u. said THC or derivative thereof is produced by a synthetic route.

15. The composition of claim 14, wherein said cannabis plant is a CBD rich strain, or wherein said cannabis plant is a THC rich strain.

16. The composition of claim 15, wherein said CBD rich strain is selected from a group consisting of Golan, Avidekel, Fedora 17, ACDC, and any combination thereof; or wherein said cannabis plant is a THC rich strain; said THC rich strain is selected from a group consisting of Everest, Black Destroyer, Critical Neville Haze, Mataro Blue, LSD OG Kush, Pineapple Chunk, Blue Monster Holk, Y Griega, Satori, Tutankhamon, and any combination thereof.

17. The composition of claim 1, wherein at least one of the following holds true:
a. said composition further comprises an additional lipophilic solvent or suspension carrier;
b. said lipophilic solvent or suspension carrier are selected from a group consisting of medium-chain triglyceride, short-chain triglyceride, medium-chain partial glyceride, polyoxyethylated fatty alcohol, polyoxyethylated fatty acid, polyoxyethylated fatty acid triglyceride or partial glyceride, ester of fatty acids with low molecular weight alcohols, a partial ester of sorbitan with fatty acids, a polyoxyethylated partial ester of sorbitan with fatty acids, a partial ester of sugars or oligomeric sugars with fatty acids, a polyethylene glycol, vegetable oil, and any combination thereof;
c. further comprising pH adjusting agents, selected from the group consisting of disodium hydrogen phosphate, sodium acetate, sodium bicarbonate, sodium phosphate tribasic, dipotassium hydrogen phosphate, phosphoric acid, acetic acid, lactic acid, fumaric acid, adipic acid, malic acid, tartaric acid, citric acid, hydrochloric acid, sulfuric acid, salts thereof, and any combination thereof;
d. further comprising osmotic agents, selected from the group consisting of glycerin, glucose and sucrose, sorbitol, sodium phosphate and any combination thereof;
e. said composition further comprising flavoring agents, selected from the group consisting of sugar, sucrose, sorbitol, sucralose, saccharin sodium, sodium cyclamate, aspartame, neotame, acesulfame potassium, stevioside, sodium chloride, D-limonene, citric acid and any combination thereof; and
f. said composition further comprising preservatives, selected from the group consisting of methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, monoglyceride, phenol, mercury components and any combination thereof.

18. The composition according to claim 1, wherein said composition is prepared by the steps of:
a. combining phospholipids, or derivatives thereof, and an oily fraction, thereby obtaining an oily phase;
b. combining said oily phase with a water phase in an emulsifier, thereby obtaining pre-emulsion; and
c. transferring said pre-emulsion into a microfluidizer, thereby obtaining a cannabis composition in the formulation of an emulsion;
further wherein said oily fraction contains about 50% cannabinoids.

19. The composition according to claim 18, comprising steps of:
a. preparing an Oil phase by mixing cannabis oil, soya phospholipids, sodium oleate and tocopherol and heating to 65° C. for 20 min;
b. preparing a water phase by mixing glycerol, EDTA and water and heating to 75° C. for 20 min;
c. adding the water phase to the oil phase and pre-mixing for 2 min at 27,000 RPM;
d. preparation of an emulsion from said mixture of step c with high-pressure homogenizer at air pressure of 150 Psi (process pressure of 45 kPsi), at room temperature;
e. measuring pH of said emulsion and optionally adjusting to pH in the range of about 5.5 to 7.5; and
f. optionally, sterilizing said emulsion.

20. A method of treating or preventing a medical condition in a subject; said method comprising administrating to the subject a therapeutically effective amount of a composition comprising phospholipids, or derivatives thereof, and an oily fraction, said composition is formulated as an emulsion, wherein said oily fraction contains about 50% cannabinoids.
